(19) United States
(12) Patent Application Publication
(10) Pub. No.: US 2006/0272041 A1
(43) Pub. Date: Nov. 30, 2006

(54) Cathepsin G (CTSG) Disruptions, Compositions and Methods Relating Thereto

(76) Inventors: Robert G. Wisotzkey, San Francisco, CA (US); Christopher J. Kirk, Millbrae, CA (US)

Correspondence Address: JOHN E. BURKE;GREENBERG TRAURIG LLP, 1200 17TH STREET, SUITE 2400, DENVER, CO, 80202, US

(21) Appl. No.: 11/457,438

(22) Filed: Jul. 13, 2006

Related U.S. Application Data
(63) Continuation-in-part of application No. 10/254,004, filed on Sep. 23, 2002.
(60) Provisional application No. 60/324,576, filed on Sep. 24, 2001.

(51) Int. Cl. A01K 67/027

(52) U.S. Cl. 800/18

(57) ABSTRACT

The present disclosure relates to compositions and methods relating to the characterization of gene function. Specifically, the present disclosure provides transgenic mice comprising disruption in a CTSG. The present disclosure also provides methods of identifying agents that modulate CTSG expression and function, useful models, and potential treatments for various disease states and disease conditions.

### RELATED APPLICATIONS

This application is a continuation-in-part of U.S. patent application Ser. No. 10/254,004, filed Sep. 23, 2002, which claims the benefit of U.S. Provisional Application 60/324,576, filed Sep. 24, 2001. The entire contents of each aforementioned provisional and nonprovisional application are incorporated herein by reference.

### FIELD OF THE INVENTION

The present disclosure relates compositions, including transgenic animals and methods relating to the characterization of gene function.

### BACKGROUND OF THE INVENTION

Proteases are proteolytic enzymes that catalyze the cleavage of peptide bonds in other proteins. The effect of such cleavage on protein molecules is diverse. In some instances, proteolytic cleavage causes the cleaved protein to become inactive. In other instances, proteolytic cleavage causes a once inactive protein to become activate. In yet other instances, proteolytic cleavage is a mechanism whereby a single polypeptide precursor is cleaved into two or more individual polypeptides.

Proteases serve to degrade invading organisms, antigen-antibody complexes and certain tissue proteins that are no longer necessary or useful to the organism. In a normally functioning organism, proteases are produced in a limited quantity and are regulated in part through the synthesis of protease inhibitors. A large number of naturally occurring protease inhibitors serve to control the endogenous proteases by limiting their reactions locally and temporally. In addition, the protease inhibitors may inhibit proteases introduced into the body by infective agents. Tissues that are particularly prone to proteolytic attack and infection, e.g. those of the respiratory tract, are rich in protease inhibitors.

Proteases, such as, for example, the serine and cysteine proteases, have been linked to apoptosis (programmed cell death), and are believed to be involved in the development and progression of numerous diseases and disorders, including Alzheimer's and Parkinson's diseases, AIDS and cancer.

One class of proteases is the serine proteases (or serine proteinases). Serine proteases are the most abundant granule constituents of several major hematopoietic cell lineages. Serine proteases are highly abundant and have strict tissue specificity. These attributes have become important phenotypic cell markers used for studies of various aspects of hematopietic cell development. Two of the major neutrophil and monocyte serine proteases in the mouse, cathepsin G and mouse protease 3 (myeloblastin) cDNAs were isolated. The full-length cDNA for mouse cathepsin G has been reported in the art. (See, e.g. Aveskogh et al., Immunogenetics 46(3):181 (1997)). As cathepsin G (CTSG) may be important in biological and disease processes, a clear need exists for further in vivo characterization, which may aid in the identification and discovery of therapeutics and treatments useful in preventing, ameliorating or correcting dysfunctions or diseases.

### SUMMARY OF THE INVENTION

The present disclosure generally relates to compositions, including transgenic animals and methods relating to the characterization, function and uses of CTSG.

The present disclosure provides transgenic cells comprising a disruption in CTSG. The transgenic cells of the present disclosure are comprised of any cells capable of undergoing homologous recombination. Preferably, the cells of the present disclosure are stem cells and more preferably, embryonic stem (ES) cells, and most preferably, murine ES cells. According to one embodiment, the transgenic cells are produced by introducing a targeting construct into a stem cell to produce a homologous recombinant, resulting in a disruption of CTSG. In another embodiment, the transgenic cells are derived from the transgenic animals described below. The cells derived from the transgenic animals includes cells that are isolated or present in a tissue or organ, and any cell lines or any progeny thereof.

The present disclosure also provides a targeting construct and methods of producing the targeting construct that when introduced into stem cells produces a homologous recombinant. In one embodiment, the targeting construct of the present disclosure comprises first and second polynucleotide sequences that are homologous to a CTSG gene. The targeting construct may also comprise a polynucleotide sequence that encodes a selectable marker that is preferably positioned between the two different homologous polynucleotide sequences in the construct. The targeting construct may also comprise other regulatory elements that can enhance homologous recombination.

The present disclosure further provides non-human transgenic animals and methods of producing such non-human transgenic animals comprising a disruption in CTSG. The transgenic animals of the present disclosure include transgenic animals that are heterozygous and homozygous for a disruption in the CTSG gene. In one aspect, the transgenic animals of the present disclosure are defective in the function of CTSG. In one aspect, the present disclosure provides a transgenic mouse comprising a disruption in CTSG gene, wherein there is no native expression of the endogenous CTSG gene.

The present disclosure also provides transgenic animals comprising a phenotype associated with having a disruption in CTSG. Preferably, the transgenic animals are rodents and, most preferably, are mice.

In one aspect of the present disclosure, a transgenic mouse having a disruption in the CTSG gene comprises a phenotype consistent with one or more symptoms of a disease associated with CTSG.

The present disclosure further provides methods of identifying an agent that modulates a phenotype associated with CTSG function or activity. Methods of identifying such agents comprise contacting a test agent with CTSG and determining whether the agent modulates CTSG.

The present disclosure also provides methods of identifying agents capable of affecting a phenotype of a transgenic animal. For example, a putative agent is administered to the transgenic animal and a response of the transgenic animal to the putative agent is measured and compared to the response of a “normal” or wild-type mouse, or alternatively compared to a transgenic animal control (without agent administration). The disclosure further provides agents identified according to such methods. The present disclosure also provides methods of identifying agents useful as therapeutic agents for treating conditions associated with a disruption or other mutation (including naturally occurring mutations) of the CTSG gene.

In one aspect, the transgenic mouse exhibits, relative to a wild-type control mouse, a physical phenotypic abnormality comprising decreased body length.

In another aspect, the transgenic mouse exhibits, relative to a wild-type control mouse, at least one histopathology phenotypic abnormality selected from the group consisting of lymphocytic infiltrate of the connective tissue of the salivary gland, degeneration of the thalamus in the cerebrum of the brain, lymphocytic infiltrate of the connective tissue of the pancreas, hyperplasia of the capsule of the adrenal gland, accessory cortical nodule of the pericapsular fat of the adrenal gland, dilation of the duct of the clitoral gland, degeneration of the brainstem, degeneration of the spinal cord, epidermal inclusion cyst of the spinal cord, predominantly lymphocytic infiltrate of the peribronchial of the lung, lymphocytic infiltrate of the lamina propia of the urinary bladder, lymphocytic infiltrate of brown adipose tissue, acute inflammation of the aortic root of the heart, arteritis of the left ventricle of the heart, predominantly lymphocytic infiltrate of connective tissue of the pancreas, predominantly lymphocytic infiltrate of the periductal of the pancreas, acute inflammation of the glandular stomach, dilation of the glands of the mucosa of the glandular stomach, hyperplasia of the glands of the mucosa of the glandular stomach, increased extramedullary hematopoiesis of the red pulp of the spleen, hyperplasia of the lymph node, aspermia of the epididymis, chronic inflammation of the epididymis, lymphocytic infiltrate of the periprostatic fat of the prostate gland, degeneration of the seminiferous tubules of the testis, lymphocytic infiltrate of the bone of the mandible, chonic inflammation of transverse skeletal muscle, fresh hemorrhage of the brainstem, degeneration of the neuropil of the brainstem, mixed inflammation of the perineurium of the sciatic nerve, degeneration of the white matter of the spinal cord, mixed inflammation of the alveoli of the lung, predominantly lymphocytic infiltrate of the vasculature of the lung, hyalin droplet in the nasal epithelium, acute inflammation of the extraocular muscles of the eye, nephropathy of the cortex of the kidney, chronic inflammation of the arcuate artery vasculature of the kidney.

In a further aspect, the transgenic mouse exhibits, relative to a wild-type control mouse, at least one hematological phenotypic abnormality selected from the group consisting of exhibited decreased mean corpuscular volume (MCV), increased neutrophils, decreased lymphocytes, and decreased basophils.

In one aspect, the transgenic mouse exhibits, relative to a wild-type control mouse, at least one serum chemistry phenotypic abnormality selected from the group consisting of increased blood urea nitrogen (BUN), increased osmolality (Osm), decreased glucose, decreased alanine aminotransferase (ALT), decreased aspartate aminotransferase (AST), abnormal cholesterol, and decreased high density lipoprotein (HDL).

In another aspect, the transgenic mouse exhibits, relative to a wild-type control mouse, at least one densitometric phenotypic abnormality selected from the group consisting of decreased bone area.

In a further aspect, the transgenic mouse exhibits, relative to a wild-type control mouse, at least one immunologic phenotypic abnormality selected from the group consisting of decreased change in body weight in response to DSS treatment in an irritable bowel syndrome assay, and increased survival in an irritable bowel syndrome assay.

In one aspect, the transgenic mouse exhibits, relative to a wild-type control mouse, at least one cytoflurometric assay (FACS) phenotypic abnormality selected from the group consisting of decreased CD4+CD25+ positive spleen cells and decreased CD8+ positive spleen cells.

One aspect of the present disclosure relates to a method of identifying a potential therapeutic agent for the treatment of a disease associated with the CTSG gene, in which the method includes the steps of: administering the potential therapeutic agent to a transgenic mouse having a disruption in a CTSG gene; and determining whether the potential therapeutic agent modulates the disease associated with the CTSG gene, wherein the modulation of the disease identifies a potential therapeutic agent for the treatment of that disease.

A further aspect of the present disclosure provides a method of identifying a potential therapeutic agent for the treatment of a disease associated with the CTSG gene, in which the method includes the steps of: contacting the potential therapeutic agent with CTSG gene product; and determining whether the potential therapeutic agent modulates that product, wherein modulation of the gene product identifies a potential therapeutic agent for the treatment of the disease associated with the CTSG gene.

The present disclosure further provides a method of identifying agents that modulate the effect or activity of CTSG. The method includes administering an effective amount of the agent to a transgenic animal, preferably a mouse. The method includes measuring a response of the transgenic animal, for example, to the agent, and comparing the response of the transgenic animal to a control animal, which may be, for example, a wild-type animal or alternatively, a transgenic animal control. Compounds that modulate the effect or activity of CTSG may also be screened against cells in cell-based assays, for example, to identify such compounds.

The disclosure also provides cell lines comprising nucleic acid sequences of a CTSG gene. Such cell lines may be capable of expressing such sequences by virtue of operable linkage to a promoter functional in the cell line. Preferably, expression of the CTSG gene sequence is under the control of an inducible promoter. Also provided are methods of identifying agents that interact with the CTSG gene, comprising the steps of contacting the CTSG gene with an agent and detecting an agent/CTSG gene complex. Such complexes can be detected by, for example, measuring expression of an operably linked detectable marker.

The disclosure further provides methods of treating diseases or conditions associated with a disruption in a CTSG gene, and more particularly, to a disruption or other alteration in the expression or function of the CTSG gene. In one embodiment, methods of the present disclosure involve treating diseases or conditions associated with a disruption or other alteration in the CTSG gene's expression or function, including administering to a subject in need, a therapeutic agent that affects CTSG expression or function. In accordance with this embodiment, the method comprises administration of a therapeutically effective amount of a natural, synthetic, semi-synthetic, or recombinant CTSG gene, CTSG gene products or fragments thereof as well as natural, synthetic, semi-synthetic or recombinant analogs.

In one aspect of the present disclosure, a therapeutic agent for treating a disease associated with the CTSG gene modulates the CTSG gene product. Another aspect of the present disclosure relates to a therapeutic agent for treating a disease associated with the CTSG gene, in which the agent is an agonist or antagonist of the CTSG gene product.

The present disclosure also provides compositions comprising or derived from ligands or other molecules or compounds that bind to or interact with CTSG, including agonists or antagonists of CTSG. Such agonists or antagonists of CTSG include antibodies and antibody mimetics, as well as other molecules that can readily be identified by routine assays and experiments well known in the art.

The present disclosure further provides methods of treating diseases or conditions associated with disrupted targeted gene expression or function, wherein the methods comprise detecting and replacing through gene therapy mutated or otherwise defective or abnormal CTSG genes.

In another embodiment, the phenotype (or phenotypic change) associated with a disruption in the CTSG gene is used to predict the likely effects and side effects of a drug that antagonizes the CTSG gene product. In this embodiment, the mouse is used to evaluate the gene as a “druggable target” i.e. to determine whether the development of drugs that target the CTSG gene product would be a worthwhile focus for pharmaceutical research.

Definitions

The following terms have the meanings ascribed to them below unless specified otherwise.

The term “gene” refers to (a) a gene containing at least one of the DNA sequences disclosed herein; (b) any DNA sequence that encodes the amino acid sequence encoded by the DNA sequences disclosed herein and/or; (c) any DNA sequence that hybridizes to the complement of the coding sequences disclosed herein. Preferably, the term includes coding as well as noncoding regions, and preferably includes all sequences necessary for normal gene expression.

The terms “polynucleotide” and “nucleic acid molecule” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-, double-stranded and triple helical molecules. “Oligonucleotide” refers to polynucleotides of between 5 and about 100 nucleotides of single- or double-stranded DNA. Oligonucleotides are also known as oligomers or oligos and may be isolated from genes, or chemically synthesized by methods known in the art. A “primer” refers to an oligonucleotide, usually single-stranded, that provides a 3′-hydroxyl end for the initiation of enzyme-mediated nucleic acid synthesis. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine.

A “fragment” of a polynucleotide is a polynucleotide comprised of at least 9 contiguous nucleotides, preferably at least 15 contiguous nucleotides and more preferably at least 45 nucleotides, of coding or non-coding sequences.

The term “gene targeting” refers to a type of homologous recombination that occurs when a fragment of genomic DNA is introduced into a mammalian cell and that fragment locates and recombines with endogenous homologous sequences.

The term “homologous recombination” refers to the exchange of DNA fragments between two DNA molecules or chromatids at the site of homologous nucleotide sequences.

The term “homologous” as used herein denotes a characteristic of a DNA sequence having at least about 70 percent sequence identity as compared to a reference sequence, typically at least about 85 percent sequence identity, preferably at least about 95 percent sequence identity, and more preferably about 98 percent sequence identity, and most preferably about 100 percent sequence identity as compared to a reference sequence. Homology can be determined using, for example, a “BLASTN” algorithm. It is understood that homologous sequences can accommodate insertions, deletions and substitutions in the nucleotide sequence. Thus, linear sequences of nucleotides can be essentially identical even if some of the nucleotide residues do not precisely correspond or align. The reference sequence may be a subset of a larger sequence, such as a portion of a gene or flanking sequence, or a repetitive portion of a chromosome.

The term “target gene” (alternatively referred to as “target gene sequence” or “target DNA sequence” or “target sequence” or “targeting DNA”) refers to any CTSG nucleic acid molecule, polynucleotide, or gene to be modified by homologous recombination, or any polynucleotide sequence homologous thereto, or orthologs thereof. The target sequence includes an intact gene, an exon or intron, a regulatory sequence or any region between genes. The target gene may comprise a portion of a CTSG gene or genetic locus in the individual's genomic DNA.

As used herein, a “variant” of a CTSG is defined as an amino acid sequence that is different by one or more amino acid substitutions. The variant may have “conservative” changes, wherein a substituted amino acid has similar structural or chemical properties, e.g. replacement of a leucine with isoleucine. More rarely, a variant may have “nonconservative” changes, e.g. replacement of a glycine with a tryptophan. Similar minor variations may also include amino acid deletions or insertions, or both. Guidance in determining which and how many amino acid residues may be substituted, inserted or deleted without abolishing biological or immunological activity may be found using computer programs well known in the art, for example, DNAStar software.

The term “active fragment” refers to a fragment of a CTSG that is biologically or immunologically active. The term “biologically active” refers to a CTSG having structural, regulatory or biochemical functions of the naturally occurring CTSG. Likewise, “immunologically active” defines the capability of the natural, recombinant or synthetic CTSG, or any oligopeptide thereof, to induce a specific immune response in appropriate animals or cells and to bind with specific antibodies.

The term “derivative”, as used herein, refers to the chemical modification of a nucleic acid sequence encoding a CTSG or the encoded CTSG. An example of such modifications would be replacement of hydrogen by an alkyl, acyl, or amino group. A nucleic acid derivative would encode a polypeptide which retains essential biological characteristics of a natural CTSG.

“Disruption” of a CTSG gene occurs when a fragment of genomic DNA locates and recombines with an endogenous homologous sequence. These sequence disruptions or modifications may include insertions, missense, frameshift, deletion, or substitutions, or replacements of DNA sequence, or any combination thereof. Insertions include the insertion of entire genes, which may be of animal, plant, fungal, insect, prokaryotic, or viral origin. Disruption, for example, can alter the normal gene product by inhibiting its production partially or completely or by enhancing the normal gene product's activity. In one embodiment, the disruption is a null disruption, wherein there is no significant expression of the CTSG gene.

The term “native expression” refers to the expression of the full-length polypeptide encoded by the CTSG gene, at expression levels present in the wild-type mouse. Thus, a disruption in which there is “no native expression” of the endogenous CTSG gene refers to a partial or complete reduction of the expression of at least a portion of a polypeptide encoded by an endogenous CTSG gene of a single cell, selected cells, or all of the cells of a mammal. The term “knockout” is a synonym for functional inactivation of the gene.

The term “construct” or “targeting construct” refers to an artificially assembled DNA segment to be transferred into a target tissue, cell line or animal. Typically, the targeting construct will include a gene or a nucleic acid sequence of particular interest, a marker gene and appropriate control sequences. As provided herein, the targeting construct of the present disclosure comprises a CTSG targeting construct. A “CTSG targeting construct” includes a DNA sequence homologous to at least one portion of an endogenous CTSG gene and is capable of producing a disruption in a CTSG gene in a host cell.

The term “transgenic cell” refers to a cell containing within its genome a CTSG gene that has been disrupted, modified, altered, or replaced completely or partially by the method of gene targeting.

The term “transgenic animal” refers to an animal that contains within its genome a specific gene that has been disrupted or otherwise modified or mutated by the method of gene targeting. “Transgenic animal” includes both the heterozygous animal (i.e., one defective allele and one wild-type allele) and the homozygous animal (i.e., two defective alleles).

As used herein, the terms “selectable marker” and “positive selection marker” refer to a gene encoding a product that enables only the cells that carry the gene to survive and/or grow under certain conditions. For example, plant and animal cells that express the introduced neomycin resistance (Neo^{r}) gene are resistant to the compound G418. Cells that do not carry the Neo^{r} gene marker are killed by G418. Other positive selection markers are known to, or are within the purview of, those of ordinary skill in the art.

A “host cell” includes an individual cell or cell culture that can be or has been a recipient for vector(s) or for incorporation of nucleic acid molecules and/or proteins. Host cells include progeny of a single host cell, and the progeny may not necessarily be completely identical (in morphology or in total DNA complement) to the original parent due to natural, accidental, or deliberate mutation. A host cell includes cells transfected with the constructs of the present disclosure.

The term “modulates” or “modulation” as used herein refers to the decrease, inhibition, reduction, amelioration, increase or enhancement of a CTSG function, expression, activity, or alternatively a phenotype associated with CTSG.

The term “ameliorates” or “amelioration” as used herein refers to a decrease, reduction or elimination of a condition, disease, disorder, or phenotype, including an abnormality or symptom.

The term “abnormality” refers to any disease, disorder, condition, or phenotype in which CTSG is implicated, including pathological conditions and behavioral observations.

### BRIEF DESCRIPTION OF THE DRAWINGS

FIG. 1 shows a polynucleotide sequence for a CTSG (SEQ ID NO:1).

FIG. 2 shows an amino acid sequence for a CTSG (SEQ ID NO:2).

FIG. 3 shows the location and extent of the disrupted portion of the CTSG gene, as well as the nucleotide sequences flanking the Neo^{r} insert in the targeting construct.

FIG. 4 shows the sequences identified as SEQ ID NO:3 and SEQ ID NO:4, which were used as the 5′- and 3′-targeting arms (including the homologous sequences) in the CTSG targeting construct, respectively.

FIG. 5 shows a table of mouse metrics data for F2N0 homozygous (−/−) and wild-type (+/+) control mice (Table 1). Statistically significant differences are highlighted in bold numbers (1-p vs. wild-type control ≧0.95).

FIG. 6 shows a table of hematology data for F2 homozygous (−/−) and wild-type (+/+) control mice (Table 2). Statistically significant differences are highlighted in bold numbers (1-p vs. wild-type control ≧0.95).

FIG. 7 shows a table of serum chemistry data for F2 homozygous (−/−) and wild-type (+/+) control mice (Table 3). Statistically significant differences are highlighted in bold numbers (1-p vs. wild-type control ≧0.95).

FIG. 8 shows a table of further serum chemistry data for F2 homozygous (−/−) and wild-type (+/+) control mice (Table 4). Statistically significant differences are highlighted in bold numbers (1-p vs. wild-type control ≧0.95).

FIG. 9 shows a table of densitometry data for F2 homozygous (−/−) and wild-type (+/+) control mice (Table 5). Statistically significant differences are highlighted in bold numbers (1-p vs. wild-type control ≧0.95).

FIG. 10A shows a graph comparing the percent change in body weight seen in homozygous (−/−) and wild-type (+/+) mice after treatment with DSS in an inflammatory bowel disease model. The homozygous treated mice show resistance to inflammatory bowel disease.

FIG. 10B shows a graph comparing the percent survival of homozygous (−/−) and wild-type (+/+) mice after treatment with DSS in an inflammatory bowel disease model.

### DETAILED DESCRIPTION OF THE INVENTION

The disclosure is based, in part, on the evaluation of the expression and role of genes and gene expression products, primarily those associated with a CTSG gene. Among other uses or applications, the disclosure permits the definition of disease pathways and the identification of diagnostically and therapeutically useful targets. For example, genes that are mutated or down-regulated under disease conditions may be involved in causing or exacerbating the disease condition. Treatments directed at up-regulating the activity of such genes or treatments that involve alternate pathways, may ameliorate the disease condition.

Generation of Targeting Construct

The targeting construct of the present disclosure may be produced using standard methods known in the art. (see, e.g. Sambrook, et al., 1989, Molecular Cloning: A Laboratory Manual, Second Edition, Cold Spring Harbor Laboratory Press, Cold Spring Harbor, N.Y.; E. N. Glover (eds.), 1985, DNA Cloning: A Practical Approach, Volumes I and II; M. J. Gait (ed.), 1984, Oligonucleotide Synthesis; B. D. Hames & S. J. Higgins (eds.), 1985, Nucleic Acid Hybridization; B. D. Hames & S. J. Higgins (eds.), 1984, Transcription and Translation; R. I. Freshney (ed.), 1986, Animal Cell Culture; Immobilized Cells and Enzymes, IRL Press, 1986; B. Perbal, 1984, A Practical Guide To Molecular Cloning; F. M. Ausubel et al., 1994, Current Protocols in Molecular Biology, John Wiley & Sons, Inc.). For example, the targeting construct may be prepared in accordance with conventional ways, where sequences may be synthesized, isolated from natural sources, manipulated, cloned, ligated, subjected to in vitro mutagenesis, primer repair, or the like. At various stages, the joined sequences may be cloned, and analyzed by restriction analysis, sequencing, or the like.

The targeting DNA can be constructed using techniques well known in the art. For example, the targeting DNA may be produced by chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g. a cloned cDNA or genomic DNA, synthetic DNA or from any of the aforementioned combination) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Moreover, the length of homology may be selected using known methods in the art. For example, selection may be based on the sequence composition and complexity of the predetermined endogenous target DNA sequence(s).

The targeting construct of the present disclosure typically comprises a first sequence homologous to a portion or region of the CTSG gene and a second sequence homologous to a second portion or region of the CTSG gene. The targeting construct may further comprise a positive selection marker, which is preferably positioned in between the first and the second DNA sequences that are homologous to a portion or region of the target DNA sequence. The positive selection marker may be operatively linked to a promoter and a polyadenylation signal.

Other regulatory sequences known in the art may be incorporated into the targeting construct to disrupt or control expression of a particular gene in a specific cell type. In addition, the targeting construct may also include a sequence coding for a screening marker, for example, green fluorescent protein (GFP), or another modified fluorescent protein.

Although the size of the homologous sequence is not critical and can range from as few as about 15-20 base pairs to as many as 100 kb, preferably each fragment is greater than about 1 kb in length, more preferably between about 1 and about 10 kb, and even more preferably between about 1 and about 5 kb. One of skill in the art will recognize that although larger fragments may increase the number of homologous recombination events in ES cells, larger fragments will also be more difficult to clone.

In one embodiment of the present disclosure, the targeting construct is prepared directly from a plasmid genomic library using the methods described in U.S. Pat. No. 6,815,185 issued Nov. 9, 2004, which is based on U.S. patent application Ser. No. 09/885,816, filed Jun. 19, 2001, which is a continuation of U.S. application Ser. No. 09/193,834, filed Nov. 17, 1998, now abandoned, which claims priority to provisional application No. 60/084,949, filed on May 11, 1998, and provisional application No. 60/084,194; and U.S. patent application Ser. No. 08/971,310, filed Nov. 17, 1997, which was converted to provisional application No. 60/084,194; the disclosure of which is incorporated herein in its entirety. Generally, a sequence of interest is identified and isolated from a plasmid library in a single step using, for example, long-range PCR. Following isolation of this sequence, a second polynucleotide that will disrupt the target sequence can be readily inserted between two regions encoding the sequence of interest. In accordance with this aspect, the construct is generated in two steps by (1) amplifying (for example, using long-range PCR) sequences homologous to the target sequence, and (2) inserting another polynucleotide (for example a selectable marker) into the PCR product so that it is flanked by the homologous sequences. Typically, the vector is a plasmid from a plasmid genomic library. The completed construct is also typically a circular plasmid.

In another embodiment, the targeting construct is designed in accordance with the regulated positive selection method described in U.S. patent application Ser. No. 09/954,483, filed Sep. 17, 2001, which is now published U.S. Patent Publication No. 20030032175, the disclosure of which is incorporated herein in its entirety. The targeting construct is designed to include a PGK-neo fusion gene having two lacO sites, positioned in the PGK promoter and an NLS-lacI gene comprising a lac repressor fused to sequences encoding the NLS from the SV40 T antigen. In another embodiment, the targeting construct may contain more than one selectable maker gene, including a negative selectable marker, such as the herpes simplex virus tk (HSV-tk) gene. The negative selectable marker may be operatively linked to a promoter and a polyadenylation signal.

In another embodiment, the targeting construct may contain more than one selectable maker gene, including a negative selectable marker, such as the herpes simplex virus tk (HSV-tk) gene. The negative selectable marker may be operatively linked to a promoter and a polyadenylation signal. (see, e.g. U.S. Pat. No. 5,464,764; U.S. Pat. No. 5,487,992; U.S. Pat. No. 5,627,059; and U.S. Pat. No. 5,631,153).

Generation of Cells and Confirmation of Homologous Recombination Events

Once an appropriate targeting construct has been prepared, the targeting construct may be introduced into an appropriate host cell using any method known in the art. Various techniques may be employed in the present disclosure, including, for example: pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g. polybrene, polyornithine, etc., or the like (see, e.g. U.S. Pat. No. 4,873,191; Van der Putten, et al., 1985, Proc. Natl. Acad. Sci., USA 82:6148-6152; Thompson, et al., 1989, Cell 56:313-321; Lo, 1983, Mol Cell. Biol. 3:1803-1814; Lavitrano, et al., 1989, Cell, 57:717-723). Various techniques for transforming mammalian cells are known in the art. (see, e.g. Gordon, 1989, Intl. Rev. Cytol., 115:171-229; Keown et al., 1989, Methods in Enzymology; Keown et al., 1990, Methods and Enzymology, Vol. 185, pp. 527-537; Mansour et al., 1988, Nature, 336:348-352).

In one aspect of the present disclosure, the targeting construct is introduced into host cells by electroporation. In this process, electrical impulses of high field strength reversibly permeabilize biomembranes allowing the introduction of the construct. The pores created during electroporation permit the uptake of macromolecules such as DNA. (see, e.g. Potter, H., et al., 1984, Proc. Nat'l. Acad. Sci. U.S.A. 81:7161-7165).

Any cell type capable of homologous recombination may be used in the practice of the present disclosure. Examples of such target cells include cells derived from vertebrates including mammals such as humans, bovine species, ovine species, murine species, simian species, and ether eucaryotic organisms such as filamentous fungi, and higher multicellular organisms such as plants.

Preferred cell types include embryonic stem (ES) cells, which are typically obtained from pre-implantation embryos cultured in vitro. (see, e.g. Evans, M. J., et al., 1981, Nature 292:154-156; Bradley, M. O., et al., 1984, Nature 309:255-258; Gossler et al., 1986, Proc. Natl. Acad. Sci. USA 83:9065-9069; and Robertson, et al., 1986, Nature 322:445-448). The ES cells are cultured and prepared for introduction of the targeting construct using methods well known to the skilled artisan. (see, e.g. Robertson, E. J. ed. “Teratocarcinomas and Embryonic Stem Cells, a Practical Approach”, IRL Press, Washington D.C., 1987; Bradley et al., 1986, Current Topics in Devel. Biol. 20:357-371; by Hogan et al., in “Manipulating the Mouse Embryo”: A Laboratory Manual, Cold Spring Harbor Laboratory Press, Cold Spring Harbor N.Y., 1986; Thomas et al., 1987, Cell 51:503; Koller et al., 1991, Proc. Natl. Acad. Sci. USA, 88:10730; Dorin et al., 1992, Transgenic Res. 1: 101; and Veis et al., 1993, Cell 75:229). The ES cells that will be inserted with the targeting construct are derived from an embryo or blastocyst of the same species as the developing embryo into which they are to be introduced. ES cells are typically selected for their ability to integrate into the inner cell mass and contribute to the germ line of an individual when introduced into the mammal in an embryo at the blastocyst stage of development. Thus, any ES cell line having this capability is suitable for use in the practice of the present disclosure.

The present disclosure may also be used to knock out or otherwise modify or disrupt genes in other cell types, such as stem cells. By way of example, stem cells may be myeloid, lymphoid, or neural progenitor and precursor cells. These cells comprising a knock out, modification or disruption of a gene may be particularly useful in the study of CTSG gene function in individual developmental pathways. Stem cells may be derived from any vertebrate species, such as mouse, rat, dog, cat, pig, rabbit, human, non-human primates and the like.

After the targeting construct has been introduced into cells, the cells in which successful gene targeting has occurred are identified. Insertion of the targeting construct into the targeted gene is typically detected by identifying cells for expression of the marker gene. In one embodiment, the cells transformed with the targeting construct of the present disclosure are subjected to treatment with an appropriate agent that selects against cells not expressing the selectable marker. Only those cells expressing the selectable marker gene survive and/or grow under certain conditions. For example, cells that express the introduced neomycin resistance gene are resistant to the compound G418, while cells that do not express the neo gene marker are killed by G418. If the targeting construct also comprises a screening marker such as GFP, homologous recombination can be identified through screening cell colonies under a fluorescent light. Cells that have undergone homologous recombination will have deleted the GFP gene and will not fluoresce.

If a regulated positive selection method is used in identifying homologous recombination events, the targeting construct is designed so that the expression of the selectable marker gene is regulated in a manner such that expression is inhibited following random integration but is permitted (derepressed) following homologous recombination. More particularly, the transfected cells are screened for expression of the neo gene, which requires that (1) the cell was successfully electroporated, and (2) lac repressor inhibition of neo transcription was relieved by homologous recombination. This method allows for the identification of transfected cells and homologous recombinants to occur in one step with the addition of a single drug.

Alternatively, a positive-negative selection technique may be used to select homologous recombinants. This technique involves a process in which a first drug is added to the cell population, for example, a neomycin-like drug to select for growth of transfected cells, i.e. positive selection. A second drug, such as FIAU is subsequently added to kill cells that express the negative selection marker, i.e. negative selection. Cells that contain and express the negative selection marker are killed by a selecting agent, whereas cells that do not contain and express the negative selection marker survive. For example, cells with non-homologous insertion of the construct express HSV thymidine kinase and therefore are sensitive to the herpes drugs such as gancyclovir (GANC) or FIAU (1-(2-deoxy 2-fluoro-B-D-arabinofluranosyl)-5-iodouracil). (see, e.g. Mansour et al., Nature 336:348-352: (1988); Capecchi, Science 244: 1288-1292, (1989); Capecchi, Trends in Genet. 5:70-76 (1989)).

Successful recombination may be identified by analyzing the DNA of the selected cells to confirm homologous recombination. Various techniques known in the art, such as PCR and/or Southern analysis may be used to confirm homologous recombination events.

Homologous recombination may also be used to disrupt genes in stem cells, and other cell types, which are not totipotent embryonic stem cells. By way of example, stem cells may be myeloid, lymphoid, or neural progenitor and precursor cells. Such transgenic cells may be particularly useful in the study of CTSG gene function in individual developmental pathways. Stem cells may be derived from any vertebrate species, such as mouse, rat, dog, cat, pig, rabbit, human, non-human primates and the like.

In cells that are not totipotent, it may be desirable to knock out both copies of the target gene using methods that are known in the art. For example, cells comprising homologous recombination at a target locus that have been selected for expression of a positive selection marker (e.g. Neo^{r}) and screened for non-random integration, can be further selected for multiple copies of the selectable marker gene by exposure to elevated levels of the selective agent (e.g. G418). The cells are then analyzed for homozygosity at the target locus. Alternatively, a second construct can be generated with a different positive selection marker inserted between the two homologous sequences. The two constructs can be introduced into the cell either sequentially or simultaneously, followed by appropriate selection for each of the positive marker genes. The final cell is screened for homologous recombination of both alleles of the target gene.

Production of Transgenic Animals

Selected cells are then injected into a blastocyst (or other stage of development suitable for the purposes of creating a viable animal, such as, for example, a morula) of an animal (e.g. a mouse) to form chimeras (see e.g. Bradley, A. in Teratocarcinomas and Embryonic Stem Cells: A Practical Approach, E. J. Robertson, ed., IRL, Oxford, pp. 113-152 (1987)). Alternatively, selected ES cells can be allowed to aggregate with dissociated mouse embryo cells to form the aggregation chimera. A chimeric embryo can then be implanted into a suitable pseudopregnant female foster animal and the embryo brought to term. Chimeric progeny harbouring the homologously recombined DNA in their germ cells can be used to breed animals in which all cells of the animal contain the homologously recombined DNA. In one embodiment, chimeric progeny mice are used to generate a mouse with a heterozygous disruption in the CTSG gene. Heterozygous transgenic mice can then be mated. It is well known in the art that typically ¼ of the offspring of such matings will have a homozygous disruption in the CTSG gene.

The heterozygous and homozygous transgenic mice can then be compared to normal, wild-type mice to determine whether disruption of the CTSG gene causes phenotypic changes, especially pathological changes. For example, heterozygous and homozygous mice may be evaluated for phenotypic changes by physical examination, necropsy, histology, clinical chemistry, complete blood count, body weight, organ weights, and cytological evaluation of bone marrow. Phenotypic changes may also comprise behavioral modifications or abnormalities.

In one embodiment, the phenotype (or phenotypic change) associated with a disruption in the CTSG gene is placed into or stored in a database. Preferably, the database includes: (i) genotypic data (e.g. identification of the disrupted gene) and (ii) phenotypic data (e.g. phenotype(s) resulting from the gene disruption) associated with the genotypic data. The database is preferably electronic. In addition, the database is preferably combined with a search tool so that the database is searchable.

Conditional Transgenic Animals

The present disclosure further contemplates conditional transgenic or knockout animals, such as those produced using recombination methods. Bacteriophage P1 Cre recombinase and flp recombinase from yeast plasmids are two non-limiting examples of site-specific DNA recombinase enzymes that cleave DNA at specific target sites (lox P sites for cre recombinase and frt sites for flp recombinase) and catalyze a ligation of this DNA to a second cleaved site. A large number of suitable alternative site-specific recombinases have been described, and their genes can be used in accordance with the method of the present disclosure. Such recombinases include the Int recombinase of bacteriophage λ (with or without Xis) (Weisberg, R. et al., in Lambda II, (Hendrix, R., et al., Eds.), Cold Spring Harbor Press, Cold Spring Harbor, N.Y., pp. 211-50 (1983), herein incorporated by reference); TpnI and the β-lactamase transposons (Mercier, et al., J. Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J. Bacteriol., 172:610-18 (1990)); the B. subtilis SpoIVC recombinase (Sato, et al., J. Bacteriol. 172:1092-98 (1990)); the Flp recombinase (Schwartz & Sadowski, J. Molec. Biol., 205:647-658 (1989); Parsons, et al., J. Biol. Chem., 265:4527-33 (1990); Golic & Lindquist, Cell, 59:499-509 (1989); Amin, et al., J. Molec. Biol., 214:55-72 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol., 205:493-500 (1989)), all herein incorporated by reference. Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al., (Mol Cell. Biochem., 92:107-16 (1990)); and Cregg & Madden (Mol. Gen. Genet., 219:320-23 (1989)), all herein incorporated by reference.

Cre has been purified to homogeneity, and its reaction with the loxP site has been extensively characterized (Abremski & Hess J. Mol. Biol. 259:1509-14 (1984), herein incorporated by reference). Cre protein has a molecular weight of 35,000 and can be obtained commercially from New England Nuclear/Du Pont. The cre gene (which encodes the Cre protein) has been cloned and expressed (Abremski, et al., Cell 32:1301-11 (1983), herein incorporated by reference). The Cre protein mediates recombination between two loxP sequences (Sternberg, et al., Cold Spring Harbor Symp. Quant. Biol. 45:297-309 (1981)), which may be present on the same or different DNA molecule. Because the internal spacer sequence of the loxP site is asymmetrical, two loxP sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al., Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

Recombinases have important application for characterizing gene function in knockout models. When the constructs described herein are used to disrupt CTSG genes, a fusion transcript can be produced when insertion of the positive selection marker occurs downstream (3′) of the translation initiation site of the CTSG gene. The fusion transcript could result in some level of protein expression with unknown consequence. It has been suggested that insertion of a positive selection marker gene can affect the expression of nearby genes. These effects may make it difficult to determine gene function after a knockout event since one could not discern whether a given phenotype is associated with the inactivation of a gene, or the transcription of nearby genes. Both potential problems are solved by exploiting recombinase activity. When the positive selection marker is flanked by recombinase sites in the same orientation, the addition of the corresponding recombinase will result in the removal of the positive selection marker. In this way, effects caused by the positive selection marker or expression of fusion transcripts are avoided.

In one embodiment, purified recombinase enzyme is provided to the cell by direct microinjection. In another embodiment, recombinase is expressed from a co-transfected construct or vector in which the recombinase gene is operably linked to a functional promoter. An additional aspect of this embodiment is the use of tissue-specific or inducible recombinase constructs that allow the choice of when and where recombination occurs. One method for practicing the inducible forms of recombinase-mediated recombination involves the use of vectors that use inducible or tissue-specific promoters or other gene regulatory elements to express the desired recombinase activity. The inducible expression elements are preferably operatively positioned to allow the inducible control or activation of expression of the desired recombinase activity. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, promoters. Vectors incorporating such promoters would only express recombinase activity in cells that express the necessary transcription factors.

Models for Disease

The cell- and animal-based systems described herein can be utilized as models for diseases. Animals of any species, including, but not limited to, mice, rats, rabbits, guinea pigs, pigs, micro-pigs, goats, and non-human primates, e.g. baboons, monkeys, and chimpanzees may be used to generate disease animal models. In addition, cells from humans may be used. These systems may be used in a variety of applications. Such assays may be utilized as part of screening strategies designed to identify agents, such as compounds that are capable of ameliorating disease symptoms. Thus, the animal- and cell-based models may be used to identify drugs, pharmaceuticals, therapies and interventions that may be effective in treating disease.

Cell-based systems may be used to identify compounds that may act to ameliorate disease symptoms. For example, such cell systems may be exposed to a compound suspected of exhibiting an ability to ameliorate disease symptoms, at a sufficient concentration and for a time sufficient to elicit such an amelioration of disease symptoms in the exposed cells. After exposure, the cells are examined to determine whether one or more of the disease cellular phenotypes has been altered to resemble a more normal or more wild-type, non-disease phenotype.

In addition, animal-based disease systems, such as those described herein, may be used to identify compounds capable of ameliorating disease symptoms. Such animal models may be used as test substrates for the identification of drugs, pharmaceuticals, therapies, and interventions that may be effective in treating a disease or other phenotypic characteristic of the animal. For example, animal models may be exposed to a compound or agent suspected of exhibiting an ability to ameliorate disease symptoms, at a sufficient concentration and for a time sufficient to elicit such an amelioration of disease symptoms in the exposed animals. The response of the animals to the exposure may be monitored by assessing the reversal of disorders associated with the disease. Exposure may involve treating mother animals during gestation of the model animals described herein, thereby exposing embryos or fetuses to the compound or agent that may prevent or ameliorate the disease or phenotype. Neonatal, juvenile, and adult animals can also be exposed.

More particularly, using the animal models of the disclosure, methods of identifying agents are provided, in which such agents can be identified on the basis of their ability to affect at least one phenotype associated with a disruption in a CTSG gene. In one embodiment, the present disclosure provides a method of identifying agents having an effect on CTSG expression or function. The method includes measuring a physiological response of the animal, for example, to the agent and comparing the physiological response of such animal to a control animal, wherein the physiological response of the animal comprising a disruption in a CTSG as compared to the control animal indicates the specificity of the agent. A “physiological response” is any biological or physical parameter of an animal that can be measured. Molecular assays (e.g. gene transcription, protein production and degradation rates), physical parameters (e.g. exercise physiology tests, measurement of various parameters of respiration, measurement of heart rate or blood pressure and measurement of bleeding time), behavioral testing, and cellular assays (e.g., immunohistochemical assays of cell surface markers, or the ability of cells to aggregate or proliferate) can be used to assess a physiological response.

Additionally, the transgenic animals and cells of the present disclosure may be utilized as models for diseases, disorders, or conditions associated with phenotypes relating to a disruption in a CTSG gene.

The present disclosure provides a unique animal model for testing and developing new treatments relating to the behavioral phenotypes. Analysis of the behavioral phenotype allows for the development of an animal model useful for testing, for instance, the efficacy of proposed genetic and pharmacological therapies for human genetic diseases, such as neurological, neuropsychological, or psychotic illnesses.

A statistical analysis of the various behaviors measured can be carried out using any conventional statistical program routinely used by those skilled in the art (such as, for example, “Analysis of Variance” or ANOVA). A “p” value of about 0.05 or less is generally considered to be statistically significant, although slightly higher p values may still be indicative of statistically significant differences. To statistically analyze abnormal behavior, a comparison is made between the behavior of a transgenic animal (or a group thereof) to the behavior of a wild-type mouse (or a group thereof), typically under certain prescribed conditions. “Abnormal behavior” as used herein refers to behavior exhibited by an animal having a disruption in the CTSG gene, e.g. transgenic animal, which differs from an animal without a disruption in the CTSG gene, e.g. wild-type mouse. Abnormal behavior consists of any number of standard behaviors that can be objectively measured (or observed) and compared. In the case of comparison, it is preferred that the change be statistically significant to confirm that there is indeed a meaningful behavioral difference between the knockout animal and the wild-type control animal. Examples of behaviors that may be measured or observed include, but are not limited to, ataxia, rapid limb movement, eye movement, breathing, motor activity, cognition, emotional behaviors, social behaviors, hyperactivity, hypersensitivity, anxiety, impaired learning, abnormal reward behavior, and abnormal social interaction, such as aggression.

A series of tests may be used to measure the behavioral phenotype of the animal models of the present disclosure, including neurological and neuropsychological tests to identify abnormal behavior. These tests may be used to measure abnormal behavior relating to, for example, learning and memory, eating, pain, aggression, sexual reproduction, anxiety, depression, schizophrenia, and drug abuse. (see, e.g., Crawley & Paylor, Hormones and Behavior 31:197-211 (1997)).

The social interaction test involves exposing a mouse to other animals in a variety of settings. The social behaviors of the animals (e.g. touching, climbing, sniffing, and mating) are subsequently evaluated. Differences in behaviors can then be statistically analyzed and compared (see, e.g. S. E. File, et al., Pharmacol. Bioch. Behav. 22:941-944 (1985); R. R. Holson, Phys. Behav. 37:239-247 (1986)). Examplary behavioral tests include the following.

The mouse startle response test typically involves exposing the animal to a sensory (typically auditory) stimulus and measuring the startle response of the animal (see, e.g. M. A. Geyer, et al., Brain Res. Bull. 25:485-498 (1990); Paylor and Crawley, Psychopharmacology 132:169-180 (1997)). A pre-pulse inhibition test can also be used, in which the percent inhibition (from a normal startle response) is measured by “cueing” the animal first with a brief low-intensity pre-pulse prior to the startle pulse.

The electric shock test generally involves exposure to an electrified surface and measurement of subsequent behaviors such as, for example, motor activity, learning, social behaviors. The behaviors are measured and statistically analyzed using standard statistical tests. (see, e.g. G. J. Kant, et al., Pharm. Bioch. Behav. 20:793-797 (1984); N. J. Leidenheimer, et al., Pharmacol. Bioch. Behav. 30:351-355 (1988)).

The tail-pinch or immobilization test involves applying pressure to the tail of the animal and/or restraining the animal's movements. Motor activity, social behavior, and cognitive behavior are examples of the areas that are measured. (see, e.g. M. Bertolucci D'Angic, et al., Neurochem. 55:1208-1214 (1990)).

The novelty test generally comprises exposure to a novel environment and/or novel objects. The animal's motor behavior in the novel environment and/or around the novel object are measured and statistically analyzed. (see, e.g. D. K. Reinstein, et al., Pharm. Bioch. Behav. 17:193-202 (1982); B. Poucet, Behav. Ne rosci. 103:1009-10016 (1989); R. R. Holson, et al., Phys. Behav. 37:231-238 (1986)). This test may be used to detect visual processing deficiencies or defects.

The learned helplessness test involves exposure to stresses, for example, noxious stimuli, which cannot be affected by the animal's behavior. The animal's behavior can be statistically analyzed using various standard statistical tests. (see, e.g. A. Leshner, et al., Behav. Neural Biol. 26:497-501 (1979)).

Alternatively, a tail suspension test may be used, in which the “immobile” time of the mouse is measured when suspended “upside-down” by its tail. This is a measure of whether the animal struggles, an indicator of depression. In humans, depression is believed to result from feelings of a lack of control over one's life or situation. It is believed that a depressive state can be elicited in animals by repeatedly subjecting them to aversive situations over which they have no control. A condition of “learned helplessness” is eventually reached, in which the animal will stop trying to change its circumstances and simply accept its fate. Animals that stop struggling sooner are believed to be more prone to depression. Studies have shown that the administration of certain antidepressant drugs prior to testing increases the amount of time that animals struggle before giving up.

The Morris water-maze test comprises learning spatial orientations in water and subsequently measuring the animal's behaviors, such as, for example, by counting the number of incorrect choices. The behaviors measured are statistically analyzed using standard statistical tests. (see, e.g. E. M. Spruijt, et al., Brain Res. 527:192-197 (1990)).

Alternatively, a Y-shaped maze may be used (see, e.g. McFarland, D. J., Pharmacology, Biochemistry and Behavior 32:723-726 (1989); Dellu, F., et al., Neurobiology of Learning and Memory 73:31-48 (2000)). The Y-maze is generally believed to be a test of cognitive ability. The dimensions of each arm of the Y-maze can be, for example, approximately 40 cm×8 cm×20 cm, although other dimensions may be used. Each arm can also have, for example, sixteen equally spaced photobeams to automatically detect movement within the arms. At least two different tests can be performed using such a Y-maze. In a continuous Y-maze paradigm, mice are allowed to explore all three arms of a Y-maze for, e.g. approximately 10 minutes. The animals are continuously tracked using photobeam detection grids, and the data can be used to measure spontaneous alteration and positive bias behavior. Spontaneous alteration refers to the natural tendency of a “normal” animal to visit the least familiar arm of a maze. An alternation is scored when the animal makes two consecutive turns in the same direction, thus representing a sequence of visits to the least recently entered arm of the maze. Position bias determines egocentrically defined responses by measuring the animal's tendency to favor turning in one direction over another. Therefore, the test can detect differences in an animal's ability to navigate on the basis of allocentric or egocentric mechanisms. The two-trial Y-maze memory test measures response to novelty and spatial memory based on a free-choice exploration paradigm. During the first trial (acquisition), the animals are allowed to freely visit two arms of the Y-maze for, e.g. approximately 15 minutes. The third arm is blocked off during this trial. The second trial (retrieval) is performed after an intertrial interval of, e.g. approximately 2 hours. During the retrieval trial, the blocked arm is opened and the animal is allowed access to all three arms for, e.g. approximately 5 minutes. Data are collected during the retrieval trial and analyzed for the number and duration of visits to each arm. Because the three arms of the maze are virtually identical, discrimination between novelty and familiarity is dependent on “environmental” spatial cues around the room relative to the position of each arm. Changes in arm entry and duration of time spent in the novel arm in a transgenic animal model may be indicative of a role of that gene in mediating novelty and recognition processes.

The passive avoidance or shuttle box test generally involves exposure to two or more environments, one of which is noxious, providing a choice to be learned by the animal. Behavioral measures include, for example, response latency, number of correct responses, and consistency of response. (see, e.g. R. Ader, et al., Psychon. Sci. 26:125-128 (1972); R. R. Holson, Phys. Behav. 37:221-230 (1986)). Alternatively, a zero-maze can be used. In a zero-maze, the animals can, for example, be placed in a closed quadrant of an elevated annular platform having, e.g. 2 open and 2 closed quadrants, and are allowed to explore for approximately 5 minutes. This paradigm exploits an approach-avoidance conflict between normal exploratory activity and an aversion to open spaces in rodents. This test measures anxiety levels and can be used to evaluate the effectiveness of anti-anxiolytic drugs. The time spent in open quadrants versus closed quadrants may be recorded automatically, with, for example, the placement of photobeams at each transition site.

The food avoidance test involves exposure to novel food and objectively measuring, for example, food intake and intake latency. The behaviors measured are statistically analyzed using standard statistical tests. (see, e.g. B. A. Campbell, et al., J. Comp. Physiol. Psychol. 67:15-22 (1969)).

The elevated plus-maze test comprises exposure to a maze, without sides, on a platform, the animal's behavior is objectively measured by counting the number of maze entries and maze learning. The behavior is statistically analyzed using standard statistical tests. (see, e.g. H. A. Baldwin, et al., Brain Res. Bull, 20:603-606 (1988)).

The stimulant-induced hyperactivity test involves injection of stimulant drugs (e.g., amphetamines, cocaine, PCP, and the like), and objectively measuring, for example, motor activity, social interactions, cognitive behavior. The animal's behaviors are statistically analyzed using standard statistical tests. (see, e.g. P. B. S. Clarke, et al., Psychopharmacology 96:511-520 (1988); P. Kuczenski, et al., J. Neuroscience 11:2703-2712 (1991)).

The self-stimulation test generally comprises providing the mouse with the opportunity to regulate electrical and/or chemical stimuli to its own brain. Behavior is measured by frequency and pattern of self-stimulation. Such behaviors are statistically analyzed using standard statistical tests. (see, e.g. S. Nassif, et al., Brain Res., 332:247-257 (1985); W. L. Isaac, et al., Behav. Neurosci. 103:345-355 (1989)).

The reward test involves shaping a variety of behaviors, e.g. motor, cognitive, and social, measuring, for example, rapidity and reliability of behavioral change, and statistically analyzing the behaviors measured. (see, e.g. L. E. Jarrard, et al., Exp. Brain Res. 61:519-530 (1986)).

The DRL (differential reinforcement to low rates of responding) performance test involves exposure to intermittent reward paradigms and measuring the number of proper responses, e.g. lever pressing. Such behavior is statistically analyzed using standard statistical tests. (see, e.g. J. D. Sinden, et al., Behav. Neurosci. 100:320-329 (1986); V. Nalwa, et al., Behav Brain Res. 17:73-76 (1985); and A. J. Normeman, et al., J. Comp. Physiol. Psych. 95:588-602 (1981)).

The spatial learning test involves exposure to a complex novel environment, measuring the rapidity and extent of spatial learning, and statistically analyzing the behaviors measured. (see, e.g. N. Pitsikas, et al., Pharm. Bioch. Behav. 38:931-934 (1991); B. poucet, et al., Brain Res. 37:269-280 (1990); D. Christie, et al., Brain Res. 37:263-268 (1990); and F. Van Haaren, et al., Behav. Neurosci. 102:481-488 (1988)). Alternatively, an open-field (of) test may be used, in which the greater distance traveled for a given amount of time is a measure of the activity level and anxiety of the animal. When the open field is a novel environment, it is believed that an approach-avoidance situation is created, in which the animal is “torn” between the drive to explore and the drive to protect itself. Because the chamber is lighted and has no places to hide other than the corners, it is expected that a “normal” mouse will spend more time in the corners and around the periphery than it will in the center where there is no place to hide. “Normal” mice will, however, venture into the central regions as they explore more and more of the chamber. It can then be extrapolated that especially anxious mice will spend most of their time in the corners, with relatively little or no exploration of the central region, whereas bold (i.e., less anxious) mice will travel a greater distance, showing little preference for the periphery versus the central region.

The visual, somatosensory and auditory neglect tests generally comprise exposure to a sensory stimulus, objectively measuring, for example, orientating responses, and statistically analyzing the behaviors measured. (see, e.g. J. M. Vargo, et al., Exp. Neurol. 102:199-209 (1988)).

The consummatory behavior test generally comprises feeding and drinking, and objectively measuring quantity of consumption. The behavior measured is statistically analyzed using standard statistical tests. (see, e.g. P. J. Fletcher, et al., Psychopharmacol. 102:301-308 (1990); M. G. Corda, et al., Proc. Nat'l Acad. Sci. USA 80:2072-2076 (1983)).

A visual discrimination test can also be used to evaluate the visual processing of an animal. One or two similar objects are placed in an open field and the animal is allowed to explore for about 5-10 minutes. The time spent exploring each object (proximity to, i.e., movement within, e.g. about 3-5 cm of the object is considered exploration of an object) is recorded. The animal is then removed from the open field, and the objects are replaced by a similar object and a novel object. The animal is returned to the open field and the percent time spent exploring the novel object over the old object is measured (again, over about a 5-10 minute span). “Normal” animals will typically spend a higher percentage of time exploring the novel object rather than the old object. If a delay is imposed between sampling and testing, the memory task becomes more hippocampal-dependent. If no delay is imposed, the task is more based on simple visual discrimination. This test can also be used for olfactory discrimination, in which the objects (preferably, simple blocks) can be sprayed or otherwise treated to hold an odor. This test can also be used to determine if the animal can make gustatory discriminations; animals that return to the previously eaten food instead of novel food exhibit gustatory neophobia.

A hot plate analgesia test can be used to evaluate an animal's sensitivity to heat or painful stimuli. For example, a mouse can be placed on an approximately 55° C. hot plate and the mouse's response latency (e.g., time to pick up and lick a hind paw) can be recorded. These responses are not reflexes, but rather “higher” responses requiring cortical involvement. This test may be used to evaluate a nociceptive disorder.

A tail-flick test may also be used to evaluate an animal's sensitivity to heat or painful stimuli. For example, a high-intensity thermal stimulus can be directed to the tail of a mouse and the mouse's response latency recorded (e.g. the time from onset of stimulation to a rapid flick/withdrawal from the heat source) can be recorded. These responses are simple nociceptive reflexive responses that are involuntary spinally mediated flexion reflexes. This test may also be sued to evaluate a nociceptive disorder.

An accelerating rotarod test may be used to measure coordination and balance in mice. Animals can be, for example, placed on a rod that acts like a rotating treadmill (or rolling log). The rotarod can be made to rotate slowly at first and then progressively faster until it reaches a speed of, e.g. approximately 60 rpm. The mice must continually reposition themselves in order to avoid falling off. The animals are preferably tested in at least three trials, a minimum of 20 minutes apart. Those mice that are able to stay on the rod the longest are believed to have better coordination and balance.

A metrazol administration test can be used to screen animals for varying susceptibilities to seizures or similar events. For example, a 5 mg/ml solution of metrazol can be infused through the tail vein of a mouse at a rate of, e.g. approximately 0.375 ml/min. The infusion will cause all mice to experience seizures, followed by death. Those mice that enter the seizure stage the soonest are believed to be more prone to seizures. Four distinct physiological stages can be recorded: soon after the start of infusion, the mice will exhibit a noticeable “twitch”, followed by a series of seizures, ending in a final tensing of the body known as “tonic extension”, which is followed by death.

CTSG Nucleic Acid Sequence and CTSG Gene Products

The present disclosure further contemplates use of the CTSG gene sequence to produce CTSG gene products. CTSG gene products may include proteins that represent functionally equivalent gene products. Such an equivalent gene product may contain deletions, additions or substitutions of amino acid residues within the amino acid sequence encoded by the gene sequences described herein, but which result in a silent change, thus producing a functionally equivalent CTSG gene product. Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent”, as utilized herein, refers to a protein capable of exhibiting a substantially similar in vivo activity as the endogenous gene products encoded by the CTSG gene sequences. Alternatively, when utilized as part of an assay, “functionally equivalent” may refer to peptides capable of interacting with other cellular or extracellular molecules in a manner substantially similar to the way in which the corresponding portion of the endogenous gene product would.

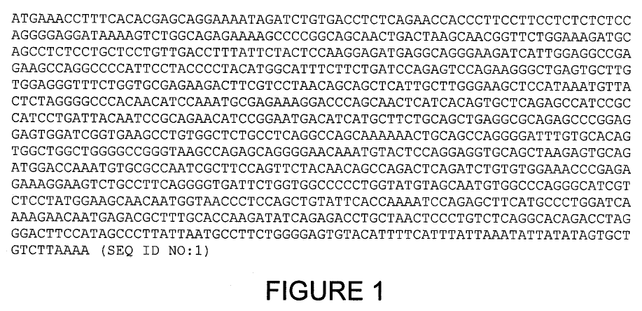

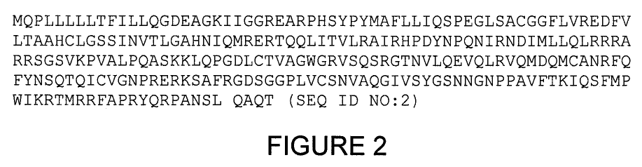

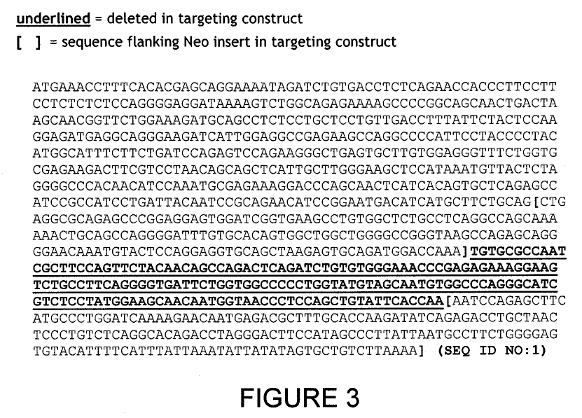

CTSG nucleic acid sequences and amino acid sequences may include: (1) the sequence shown in FIG. 1 (SEQ ID NO:1) or identified in Genebank as Accession No. NM_{—}007800; GI:6681082; (2) the CTSG polypeptide as shown in FIG. 2 (SEQ ID NO:2) or identified in GenBank Accession No.: NP_{—}031826.1 GI:6681083; or (3) any homologues, orthologs, variants, derivatives, active fragments or mutants of CTSG.

“Percent identity” or “% identity” refers to the percentage of sequence similarity found in a comparison of two or more amino acid or nucleic acid sequences. Percent identity can be determined electronically, e.g., by using the MegAlign™ program (DNASTAR, Inc., Madison Wis.). The MegAlign™ program can create alignments between two or more sequences according to different methods, e.g., the clustal method. (See, e.g., Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between nucleic acid sequences can also be counted or calculated by other methods known in the art, e.g., the Jotun Hein method. (See, e.g. Hein, J. (1990) Methods Enzymol. 183:626-645.) Identity between sequences can also be determined by other methods known in the art, e.g., by varying hybridization conditions.

Substantially purified variants, preferably, having at least 90% sequence identity to a CTSG or to a fragment of CTSG may be used in the methods of identifying agents that modulate CTSG or alternatively a phenotype associated with CTSG function as disclosed by the present disclosure.

Isolated and purified polynucleotides which hybridize under stringent conditions to CTSG or a fragment of CTSG, as well as an isolated and purified CTSG polynucleotide complementary to a CTSG polynucleotide encoding a CTSG amino acid sequence or a fragment thereof may be used in methods of identifying agents that modulate CTSG or alternatively a phenotype associated with CTSG function as disclosed by the present disclosure.

“Stringent conditions” refers to conditions which permit hybridization between polynucleotides and CTSG polynucleotides. Stringent conditions can be defined by salt concentration, the concentration of organic solvent, e.g., formamide, temperature, and other conditions well known in the art. In particular, stringency can be increased by reducing the concentration of salt, increasing the concentration of formamide, or raising the hybridization temperature. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30.degree. C., more preferably of at least about 37.degree. C., and most preferably of at least about 42.degree. C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30.degree. C. in 750 mM NaCl, 75 mM trisodium citrate, and 1% SDS. In a more preferred embodiment, hybridization will occur at 37.degree. C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42.degree. C. in 250 mM NaCl, 25 mM trisodium citrate, 1% SDS, 50% formamide, and 200 .mu.g/ml ssDNA. Useful variations on these conditions will be readily apparent to those skilled in the art.

Other protein products useful according to the methods of the disclosure are peptides derived from or based on the CTSG gene products produced by recombinant or synthetic means (derived peptides).

CTSG gene products may be produced by recombinant DNA technology using techniques well known in the art. Thus, methods for preparing the gene polypeptides and peptides of the disclosure by expressing nucleic acids encoding gene sequences are described herein. Methods that are well known to those skilled in the art can be used to construct expression vectors containing gene protein coding sequences and appropriate transcriptional/translational control signals. These methods include, for example, in vitro recombinant DNA techniques, synthetic techniques and in vivo recombination/genetic recombination (see, e.g. Sambrook, et al., 1989, supra, and Ausubel, et al., 1989, supra). Alternatively, RNA capable of encoding gene protein sequences may be chemically synthesized using, for example, automated synthesizers (see, e.g. Oligonucleotide Synthesis: A Practical Approach, Gait, M. J. ed., IRL Press, Oxford (1984)).

A variety of host-expression vector systems may be utilized to express the gene coding sequences of the disclosure. Such host-expression systems represent vehicles by which the coding sequences of interest may be produced and subsequently purified, but also represent cells that may, when transformed or transfected with the appropriate nucleotide coding sequences, exhibit the gene protein of the disclosure in situ. These include but are not limited to microorganisms such as bacteria (e.g. E. coli, B. subtilis) transformed with recombinant bacteriophage DNA, plasmid DNA or cosmid DNA expression vectors containing gene protein coding sequences; yeast (e.g. Saccharomyces, Pichia) transformed with recombinant yeast expression vectors containing the gene protein coding sequences; insect cell systems infected with recombinant virus expression vectors (e.g. baculovirus) containing the gene protein coding sequences; plant cell systems infected with recombinant virus expression vectors (e.g. cauliflower mosaic virus, CaMV; tobacco mosaic virus, TMV) or transformed with recombinant plasmid expression vectors (e.g. Ti plasmid) containing gene protein coding sequences; or mammalian cell systems (e.g. COS, CHO, BHK, 293, 3T3) harboring recombinant expression constructs containing promoters derived from the genome of mammalian cells (e.g. metallothionine promoter) or from mammalian viruses (e.g. the adenovirus late promoter; the vaccinia virus 7.5 K promoter).

In bacterial systems, a number of expression vectors may be advantageously selected depending upon the use intended for the gene protein being expressed. For example, when a large quantity of such a protein is to be produced, for the generation of antibodies or to screen peptide libraries, for example, vectors that direct the expression of high levels of fusion protein products that are readily purified may be desirable. Such vectors include, but are not limited, to the E. coli expression vector pUR278 (Ruther et al., EMBO J., 2:1791-94 (1983)), in which the gene protein coding sequence may be ligated individually into the vector in frame with the lac Z coding region so that a fusion protein is produced; pIN vectors (Inouye & Inouye, Nucleic Acids Res., 13:3101-09 (1985); Van Heeke et al., J. Biol. Chem., 264:5503-9 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned CTSG gene protein can be released from the GST moiety.

In one embodiment, full length cDNA sequences are appended with in-frame Bam HI sites at the amino terminus and Eco RI sites at the carboxyl terminus using standard PCR methodologies (Innis, et al. (eds) PCR Protocols: A Guide to Methods and Applications, Academic Press, San Diego (1990)) and ligated into the pGEX-2TK vector (Pharmacia, Uppsala, Sweden). The resulting cDNA construct contains a kinase recognition site at the amino terminus for radioactive labeling and glutathione S-transferase sequences at the carboxyl terminus for affinity purification (Nilsson, et al., EMBO J., 4: 1075-80 (1985); Zabeau et al., EMBO J., 1: 1217-24 (1982)).

In an insect system, Autographa californica nuclear polyhedrosis virus (AcNPV) is used as a vector to express foreign genes. The virus grows in Spodoptera frugiperda cells. The gene coding sequence may be cloned individually into non-essential regions (for example the polyhedrin gene) of the virus and placed under control of an AcNPV promoter (for example the polyhedrin promoter). Successful insertion of gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see, e.g. Smith, et al., J. Virol. 46: 584-93 (1983); U.S. Pat. No. 4,745,051).

In mammalian host cells, a number of viral-based expression systems may be utilized. In cases where an adenovirus is used as an expression vector, the gene coding sequence of interest may be ligated to an adenovirus transcription/translation control complex, e.g. the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing gene protein in infected hosts. (e.g. see Logan et al., Proc. Natl. Acad. Sci. USA, 81:3655-59 (1984)). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter, et al., Methods in Enzymol., 153:516-44 (1987)).

In addition, a host cell strain may be chosen that modulates the expression of the inserted sequences, or modifies and processes the gene product in the specific fashion desired. Such modifications (e.g. glycosylation) and processing (e.g. cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, etc.

For long-term, high-yield production of recombinant proteins, stable expression is preferred. For example, cell lines that stably express the gene protein may be engineered. Rather than using expression vectors that contain viral origins of replication, host cells can be transformed with DNA controlled by appropriate expression control elements (e.g. promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells that stably integrate the plasmid into their chromosomes and grow, to form foci, which in turn can be cloned and expanded into cell lines. This method may advantageously be used to engineer cell lines that express the gene protein. Such engineered cell lines may be particularly useful in screening and evaluation of compounds that affect the endogenous activity of the gene protein.

In one embodiment, timing and/or quantity of expression of the recombinant protein can be controlled using an inducible expression construct. Inducible constructs and systems for inducible expression of recombinant proteins will be well known to those skilled in the art. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, particularly HIV, promoters. In one in embodiment, a Tet inducible gene expression system is utilized. (Gossen et al., Proc. Natl. Acad. Sci. USA, 89:5547-51 (1992); Gossen, et al., Science, 268:1766-69 (1995)). Tet Expression Systems are based on two regulatory elements derived from the tetracycline-resistance operon of the E. coli Tn10 transposon—the tetracycline repressor protein (TetR) and the tetracycline operator sequence (tetO) to which TetR binds. Using such a system, expression of the recombinant protein is placed under the control of the tetO operator sequence and transfected or transformed into a host cell. In the presence of TetR, which is co-transfected into the host cell, expression of the recombinant protein is repressed due to binding of the TetR protein to the tetO regulatory element. High-level, regulated gene expression can then be induced in response to varying concentrations of tetracycline (Tc) or Tc derivatives such as doxycycline (Dox), which compete with tetO elements for binding to TetR. Constructs and materials for tet inducible gene expression are available commercially from CLONTECH Laboratories, Inc., Palo Alto, Calif.

When used as a component in an assay system, the gene protein may be labeled, either directly or indirectly, to facilitate detection of a complex formed between the gene protein and a test substance. Any of a variety of suitable labeling systems may be used including but not limited to radioisotopes such as ¹²⁵I; enzyme labeling systems that generate a detectable calorimetric signal or light when exposed to substrate; and fluorescent labels. Where recombinant DNA technology is used to produce the gene protein for such assay systems, it may be advantageous to engineer fusion proteins that can facilitate labeling, immobilization and/or detection.

Indirect labeling involves the use of a protein, such as a labeled antibody, which specifically binds to the gene product. Such antibodies include but are not limited to polyclonal, monoclonal, chimeric, single chain, Fab fragments and fragments produced by a Fab expression library.

Production of Antibodies

Described herein are methods for the production of antibodies capable of specifically recognizing one or more epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a CTSG gene in a biological sample, or, alternatively, as a method for the inhibition of abnormal CTSG gene activity. Thus, such antibodies may be utilized as part of disease treatment methods, and/or may be used as part of diagnostic techniques whereby patients may be tested for abnormal levels of CTSG gene proteins, or for the presence of abnormal forms of such proteins.

For the production of antibodies, various host animals may be immunized by injection with the CTSG gene, its expression product or a portion thereof. Such host animals may include but are not limited to rabbits, mice, rats, goats and chickens, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as CTSG gene product, or an antigenic functional derivative thereof. For the production of polyclonal antibodies, host animals such as those described above, may be immunized by injection with gene product supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to the hybridoma technique of Köhler and Milstein, Nature, 256:495-7 (1975); and U.S. Pat. No. 4,376,110), the human B-cell hybridoma technique (Kosbor, et al., Immunology Today, 4:72 (1983); Cote, et al., Proc. Natl. Acad. Sci. USA, 80:2026-30 (1983)), and the EBV-hybridoma technique (Cole, et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the mAb of this disclosure may be cultivated in vitro or in vivo. Production of high titers of mAbs in vivo makes this the presently preferred method of production.

In addition, techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce gene-single chain antibodies. Single chain antibodies are typically formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed (Huse, et al., Science, 246:1275-81 (1989)) to allow rapid and easy identification of monoclonal Fab fragments with the desired specificity.

Screening Methods

Various animal-derived “preparations,” including cells and tissues, as well as cell-free extracts, homogenates, fractions and purified proteins, may be used to determine whether a particular agent is capable of modulating an activity of a CTSG or a phenotype associated therewith. For example, such preparations may be generated according to methods well known in the art from the tissues or organs of wild-type and knockout animals. Wild-type, but not knockout, preparations will contain endogenous CTSG, as well as the native activities, interactions and effects of the CTSG. Thus, when knockout and wild-type preparations are contacted with a test agent in parallel, the ability of the test agent to modulate CTSG, or a phenotype associated therewith, can be determined. Agents capable of modulating an activity of a CTSG or a phenotype associated therewith are identified as those that modulate wild-type, but not knockout, preparations. Modulation may be detected, for example, as the ability of the agent to interact with a preparation, thereby indicating interaction with the gene product itself or a product thereof. Alternatively, the agent may affect a structural, metabolic or biochemical feature of the preparation, such as enzymatic activity of the preparation related to the CTSG. An inclusive discussion of the events for which modulation by a test agent may be observed is beyond the scope of this application, but will be well known by those skilled in the art.

The present disclosure may be employed in a process for screening for agents such as agonists, i.e., agents that bind to and activate CTSG polypeptides, or antagonists, i.e., inhibit the activity or interaction of CTSG polypeptides with its ligand. Thus, polypeptides of the disclosure may also be used to assess the binding of small molecule substrates and ligands in, for example, cells, cell-free preparations, chemical libraries, and natural product mixtures as known in the art. Any methods routinely used to identify and screen for agents that can modulate receptors may be used in accordance with the present disclosure.

The present disclosure provides methods for identifying and screening for agents that modulate CTSG expression or function. More particularly, cells that contain and express CTSG gene sequences may be used to screen for therapeutic agents. Such cells may include non-recombinant monocyte cell lines, such as U937 (ATCC# CRL-1593), THP-1 (ATCC# TIB-202), and P388D1 (ATCC# TIB-63); endothelial cells such as HUVEC's and bovine aortic endothelial cells (BAEC's); as well as generic mammalian cell lines such as HeLa cells and COS cells, e.g. COS-7 (ATCC# CRL-1651). Further, such cells may include recombinant, transgenic cell lines. For example, the transgenic mice of the disclosure may be used to generate cell lines, containing one or more cell types involved in a disease, that can be used as cell culture models for that disorder. While cells, tissues, and primary cultures derived from the disease transgenic animals of the disclosure may be utilized, the generation of continuous cell lines is preferred. For examples of techniques that may be used to derive a continuous cell line from the transgenic animals, see Small, et al., Mol. Cell Biol., 5:642-48 (1985).

CTSG gene sequences may be introduced into and overexpressed in, the genome of the cell of interest. In order to overexpress a CTSG gene sequence, the coding portion of the CTSG gene sequence may be ligated to a regulatory sequence that is capable of driving gene expression in the cell type of interest. Such regulatory regions will be well known to those of skill in the art, and may be utilized in the absence of undue experimentation. CTSG gene sequences may also be disrupted or underexpressed. Cells having CTSG gene disruptions or underexpressed CTSG gene sequences may be used, for example, to screen for agents capable of affecting alternative pathways that compensate for any loss of function attributable to the disruption or underexpression.

In vitro systems may be designed to identify compounds capable of binding the CTSG gene products. Such compounds may include, but are not limited to, peptides made of D- and/or L-configuration amino acids (in, for example, the form of random peptide libraries; (see e.g. Lam, et al., Nature, 354:82-4 (1991)), phosphopeptides (in, for example, the form of random or partially degenerate, directed phosphopeptide libraries; see, e.g. Songyang, et al., Cell, 72:767-78 (1993)), antibodies, and small organic or inorganic molecules. Compounds identified may be useful, for example, in modulating the activity of CTSG gene proteins, preferably mutant CTSG gene proteins; elaborating the biological function of the CTSG gene protein; or screening for compounds that disrupt normal CTSG gene interactions or themselves disrupt such interactions.

The principle of the assays used to identify compounds that bind to the CTSG gene protein involves preparing a reaction mixture of the CTSG gene protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring the CTSG gene protein or the test substance onto a solid phase and detecting target protein/test substance complexes anchored on the solid phase at the end of the reaction. In one embodiment of such a method, the CTSG gene protein may be anchored onto a solid surface, and the test compound, which is not anchored, may be labeled, either directly or indirectly.

In practice, microtitre plates are conveniently utilized. The anchored component may be immobilized by non-covalent or covalent attachments. Non-covalent attachment may be accomplished simply by coating the solid surface with a solution of the protein and drying. Alternatively, an immobilized antibody, preferably a monoclonal antibody, specific for the protein may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g. by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g. using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g. using an immobilized antibody specific for CTSG gene product or the test compound to anchor any complexes formed in solution, and a labeled antibody specific for the other component of the possible complex to detect anchored complexes.

Compounds that are shown to bind to a particular CTSG gene product through one of the methods described above can be further tested for their ability to elicit a biochemical response from the CTSG gene protein. Agonists, antagonists and/or inhibitors of the expression product can be identified utilizing assays well known in the art.

Antisense, Ribozymes, and Antibodies

Other agents that may be used as therapeutics include the CTSG gene, its expression product(s) and functional fragments thereof. Additionally, agents that reduce or inhibit mutant CTSG gene activity may be used to ameliorate disease symptoms. Such agents include antisense, ribozyme, and triple helix molecules. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g. between the −10 and +10 regions of the CTSG gene nucleotide sequence of interest, are preferred.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. The composition of ribozyme molecules must include one or more sequences complementary to the CTSG gene mRNA, and must include the well known catalytic sequence responsible for mRNA cleavage. For this sequence, see U.S. Pat. No. 5,093,246, which is incorporated by reference herein in its entirety. As such within the scope of the disclosure are engineered hammerhead motif ribozyme molecules that specifically and efficiently catalyze endonucleolytic cleavage of RNA sequences encoding CTSG gene proteins.

Specific ribozyme cleavage sites within any potential RNA target are initially identified by scanning the molecule of interest for ribozyme cleavage sites that include the following sequences, GUA, GUU and GUC. Once identified, short RNA sequences of between 15 and 20 ribonucleotides corresponding to the region of the CTSG gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

It is possible that the antisense, ribozyme, and/or triple helix molecules described herein may reduce or inhibit the transcription (triple helix) and/or translation (antisense, ribozyme) of mRNA produced by both normal and mutant CTSG gene alleles. In order to ensure that substantially normal levels of CTSG gene activity are maintained, nucleic acid molecules that encode and express CTSG polypeptides exhibiting normal activity may be introduced into cells that do not contain sequences susceptible to whatever antisense, ribozyme, or triple helix treatments are being utilized. Alternatively, it may be preferable to coadminister normal CTSG protein into the cell or tissue in order to maintain the requisite level of cellular or tissue CTSG gene activity.

Anti-sense RNA and DNA, ribozyme, and triple helix molecules of the disclosure may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies that are both specific for CTSG protein, and in particular, the mutant CTSG protein, and interfere with its activity may be used to inhibit mutant CTSG gene function. Such antibodies may be generated against the proteins themselves or against peptides corresponding to portions of the proteins using standard techniques known in the art and as also described herein. Such antibodies include but are not limited to polyclonal, monoclonal, Fab fragments, single chain antibodies, chimeric antibodies, antibody mimetics, etc.

In instances where the CTSG protein is intracellular and whole antibodies are used, internalizing antibodies may be preferred. However, lipofectin liposomes may be used to deliver the antibody or a fragment of the Fab region that binds to the CTSG gene epitope into cells. Where fragments of the antibody are used, the smallest inhibitory fragment that binds to the target or expanded target protein's binding domain is preferred. For example, peptides having an amino acid sequence corresponding to the domain of the variable region of the antibody that binds to the CTSG protein may be used. Such peptides may be synthesized chemically or produced via recombinant DNA technology using methods well known in the art (see, e.g. Creighton, Proteins: Structures and Molecular Principles (1984) W.H. Freeman, New York 1983, supra; and Sambrook, et al., 1989, supra). Alternatively, single chain neutralizing antibodies that bind to intracellular CTSG gene epitopes may also be administered. Such single chain antibodies may be administered, for example, by expressing nucleotide sequences encoding single-chain antibodies within the target cell population by utilizing, for example, techniques such as those described in Marasco, et al., Proc. Natl. Acad. Sci. USA, 90:7889-93 (1993).

RNA sequences encoding CTSG protein may be directly administered to a patient exhibiting disease symptoms, at a concentration sufficient to produce a level of CTSG protein such that disease symptoms are ameliorated. Patients may be treated by gene replacement therapy. One or more copies of a normal CTSG gene, or a portion of the gene that directs the production of a normal CTSG protein with CTSG gene function, may be inserted into cells using vectors that include, but are not limited to adenovirus, adeno-associated virus, and retrovirus vectors, in addition to other particles that introduce DNA into cells, such as liposomes. Additionally, techniques such as those described above may be utilized for the introduction of normal CTSG gene sequences into human cells.

Cells, preferably autologous cells, containing normal CTSG gene expressing gene sequences may then be introduced or reintroduced into the patient at positions that allow for the amelioration of disease symptoms.

Pharmaceutical Compositions, Effective Dosages, and Routes of Administration

The identified compounds that inhibit target mutant gene expression, synthesis and/or activity can be administered to a patient at therapeutically effective doses to treat or ameliorate the disease. A therapeutically effective dose refers to that amount of the compound sufficient to result in amelioration of symptoms of the disease.

Toxicity and therapeutic efficacy of such compounds can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present disclosure may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral, topical, subcutaneous, intraperitoneal, intraveneous, intrapleural, intraoccular, intraarterial, or rectal administration. It is also contemplated that pharmaceutical compositions may be administered with other products that potentiate the activity of the compound and optionally, may include other therapeutic ingredients.

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present disclosure are conveniently delivered in the form of an aerosol spray presentation from pressurized packs or a nebuliser, with the use of a suitable propellant, e.g. dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g. by bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g. sterile pyrogen-free water, before use.

The compounds may also be formulated in rectal compositions such as suppositories or retention enemas, e.g. containing conventional suppository bases such as cocoa butter or other glycerides. Oral ingestion is possibly the easiest method of taking any medication. Such a route of administration, is generally simple and straightforward and is frequently the least inconvenient or unpleasant route of administration from the patient's point of view. However, this involves passing the material through the stomach, which is a hostile environment for many materials, including proteins and other biologically active compositions. As the acidic, hydrolytic and proteolytic environment of the stomach has evolved efficiently to digest proteinaceous materials into amino acids and oligopeptides for subsequent anabolism, it is hardly surprising that very little or any of a wide variety of biologically active proteinaceous material, if simply taken orally, would survive its passage through the stomach to be taken up by the body in the small intestine. The result, is that many proteinaceous medicaments must be taken in through another method, such as parenterally, often by subcutaneous, intramuscular or intravenous injection.

Pharmaceutical compositions may also include various buffers (e.g. Tris, acetate, phosphate), solubilizers (e.g. Tween, Polysorbate), carriers such as human serum albumin, preservatives (thimerosol, benzyl alcohol) and anti-oxidants such as ascorbic acid in order to stabilize pharmaceutical activity. The stabilizing agent may be a detergent, such as tween-20, tween-80, NP-40 or Triton X-100. EBP may also be incorporated into particulate preparations of polymeric compounds for controlled delivery to a patient over an extended period of time. A more extensive survey of components in pharmaceutical compositions is found in Remington's Pharmaceutical Sciences, 18th ed., A. R. Gennaro, ed., Mack Publishing, Easton, Pa. (1990).

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Diagnostics

A variety of methods may be employed to diagnose disease conditions associated with the CTSG gene. Specifically, reagents may be used, for example, for the detection of the presence of CTSG gene mutations, or the detection of either over- or under-expression of CTSG gene mRNA.

According to the diagnostic and prognostic method of the present disclosure, alteration of the wild-type CTSG gene locus is detected. In addition, the method can be performed by detecting the wild-type CTSG gene locus and confirming the lack of a predisposition or neoplasia. “Alteration of a wild-type gene” encompasses all forms of mutations including deletions, insertions and point mutations in the coding and noncoding regions. Deletions may be of the entire gene or only a portion of the gene. Point mutations may result in stop codons, frameshift mutations or amino acid substitutions. Somatic mutations are those that occur only in certain tissues, e.g. in tumor tissue, and are not inherited in the germline. Germline mutations can be found in any of a body's tissues and are inherited. If only a single allele is somatically mutated, an early neoplastic state may be indicated. However, if both alleles are mutated, then a late neoplastic state may be indicated. The finding of gene mutations thus provides both diagnostic and prognostic information. a CTSG gene allele that is not deleted (e.g. that found on the sister chromosome to a chromosome carrying a CTSG gene deletion) can be screened for other mutations, such as insertions, small deletions, and point mutations. Mutations found in tumor tissues may be linked to decreased expression of the CTSG gene product. However, mutations leading to non-functional gene products may also be linked to a cancerous state. Point mutational events may occur in regulatory regions, such as in the promoter of the gene, leading to loss or diminution of expression of the mRNA. Point mutations may also abolish proper RNA processing, leading to loss of expression of the CTSG gene product, or a decrease in mRNA stability or translation efficiency.

One test available for detecting mutations in a candidate locus is to directly compare genomic target sequences from cancer patients with those from a control population. Alternatively, one could sequence messenger RNA after amplification, e.g. by PCR, thereby eliminating the necessity of determining the exon structure of the candidate gene. Mutations from cancer patients falling outside the coding region of the CTSG gene can be detected by examining the non-coding regions, such as introns and regulatory sequences near or within the CTSG gene. An early indication that mutations in noncoding regions are important may come from Northern blot experiments that reveal messenger RNA molecules of abnormal size or abundance in cancer patients as compared to control individuals.

The methods described herein may be performed, for example, by utilizing pre-packaged diagnostic kits comprising at least one specific gene nucleic acid or anti-gene antibody reagent described herein, which may be conveniently used, e.g. in clinical settings, to diagnose patients exhibiting disease symptoms or at risk for developing disease.

Any cell type or tissue, including brain, cortex, subcortical region, cerebellum, brainstem, olfactory bulb, spinal cord, eye, Harderian gland, heart, lung, liver, pancreas, kidney, spleen, thymus, lymph nodes, bone marrow, skin, gallbladder, urinary bladder, pituitary gland, adrenal gland, salivary gland, skeletal muscle, tongue, stomach, small intestine, large intestine, cecum, testis, epididymis, seminal vesicle, coagulating gland, prostate gland, ovary, uterus and white fat, in which the gene is expressed may be utilized in the diagnostics described below.

DNA or RNA from the cell type or tissue to be analyzed may easily be isolated using procedures that are well known to those in the art. Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, PCR In Situ Hybridization: Protocols and Applications, Raven Press, N.Y. (1992)).

Gene nucleotide sequences, either RNA or DNA, may, for example, be used in hybridization or amplification assays of biological samples to detect disease-related gene structures and expression. Such assays may include, but are not limited to, Southern or Northern analyses, restriction fragment length polymorphism assays, single stranded conformational polymorphism analyses, in situ hybridization assays, and polymerase chain reaction analyses. Such analyses may reveal both quantitative aspects of the expression pattern of the gene, and qualitative aspects of the gene expression and/or gene composition. That is, such aspects may include, for example, point mutations, insertions, deletions, chromosomal rearrangements, and/or activation or inactivation of gene expression.

Preferred diagnostic methods for the detection of gene-specific nucleic acid molecules may involve for example, contacting and incubating nucleic acids, derived from the cell type or tissue being analyzed, with one or more labeled nucleic acid reagents under conditions favorable for the specific annealing of these reagents to their complementary sequences within the nucleic acid molecule of interest. Preferably, the lengths of these nucleic acid reagents are at least 9 to 30 nucleotides. After incubation, all non-annealed nucleic acids are removed from the nucleic acid:fingerprint molecule hybrid. The presence of nucleic acids from the fingerprint tissue that have hybridized, if any such molecules exist, is then detected. Using such a detection scheme, the nucleic acid from the tissue or cell type of interest may be immobilized, for example, to a solid support such as a membrane, or a plastic surface such as that on a microtitre plate or polystyrene beads. In this case, after incubation, non-annealed, labeled nucleic acid reagents are easily removed. Detection of the remaining, annealed, labeled nucleic acid reagents is accomplished using standard techniques well-known to those in the art.

Alternative diagnostic methods for the detection of gene-specific nucleic acid molecules may involve their amplification, e.g. by PCR (the experimental embodiment set forth in Mullis U.S. Pat. No. 4,683,202 (1987)), ligase chain reaction (Barany, Proc. Natl. Acad. Sci. USA, 88:189-93 (1991)), self sustained sequence replication (Guatelli, et al., Proc. Natl. Acad. Sci. USA, 87:1874-78 (1990)), transcriptional amplification system (Kwoh, et al., Proc. Natl. Acad. Sci. USA, 86:1173-77 (1989)), Q-Beta Replicase (Lizardi et al., Bio/Technology, 6:1197 (1988)), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment of such a detection scheme, a cDNA molecule is obtained from an RNA molecule of interest (e.g. by reverse transcription of the RNA molecule into cDNA). Cell types or tissues from which such RNA may be isolated include any tissue in which wild-type fingerprint gene is known to be expressed, including, but not limited, to brain, cortex, subcortical region, cerebellum, brainstem, olfactory bulb, spinal cord, eye, Harderian gland, heart, lung, liver, pancreas, kidney, spleen, thymus, lymph nodes, bone marrow, skin, gallbladder, urinary bladder, pituitary gland, adrenal gland, salivary gland, skeletal muscle, tongue, stomach, small intestine, large intestine, cecum, testis, epididymis, seminal vesicle, coagulating gland, prostate gland, ovary, uterus and white fat. A sequence within the cDNA is then used as the template for a nucleic acid amplification reaction, such as a PCR amplification reaction, or the like. The nucleic acid reagents used as synthesis initiation reagents (e.g. primers) in the reverse transcription and nucleic acid amplification steps of this method may be chosen from among the gene nucleic acid reagents described herein. The preferred lengths of such nucleic acid reagents are at least 15-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Antibodies directed against wild-type or mutant gene peptides may also be used as disease diagnostics and prognostics. Such diagnostic methods, may be used to detect abnormalities in the level of gene protein expression, or abnormalities in the structure and/or tissue, cellular, or subcellular location of fingerprint gene protein. Structural differences may include, for example, differences in the size, electronegativity, or antigenicity of the mutant fingerprint gene protein relative to the normal fingerprint gene protein.

Protein from the tissue or cell type to be analyzed may easily be detected or isolated using techniques that are well known to those of skill in the art, including but not limited to western blot analysis. For a detailed explanation of methods for carrying out western blot analysis, see Sambrook, et al. (1989) supra, at Chapter 18. The protein detection and isolation methods employed herein may also be such as those described in Harlow and Lane, for example, (Antibodies: A Laboratory Manual, Cold Spring Harbor Laboratory Press, Cold Spring Harbor, N.Y. (1988)).

Preferred diagnostic methods for the detection of wild-type or mutant gene peptide molecules may involve, for example, immunoassays wherein fingerprint gene peptides are detected by their interaction with an anti-fingerprint gene-specific peptide antibody.

For example, antibodies, or fragments of antibodies useful in the present disclosure may be used to quantitatively or qualitatively detect the presence of wild-type or mutant gene peptides. This can be accomplished, for example, by immunofluorescence techniques employing a fluorescently labeled antibody (see below) coupled with light microscopic, flow cytometric, or fluorimetric detection. Such techniques are especially preferred if the fingerprint gene peptides are expressed on the cell surface.

The antibodies (or fragments thereof) useful in the present disclosure may, additionally, be employed histologically, as in immunofluorescence or immunoelectron microscopy, for in situ detection of fingerprint gene peptides. In situ detection may be accomplished by removing a histological specimen from a patient, and applying thereto a labeled antibody of the present disclosure. The antibody (or fragment) is preferably applied by overlaying the labeled antibody (or fragment) onto a biological sample. Through the use of such a procedure, it is possible to determine not only the presence of the fingerprint gene peptides, but also their distribution in the examined tissue. Using the present disclosure, those of ordinary skill will readily perceive that any of a wide variety of histological methods (such as staining procedures) can be modified in order to achieve such in situ detection.

Immunoassays for wild-type, mutant, or expanded fingerprint gene peptides typically comprise incubating a biological sample, such as a biological fluid, a tissue extract, freshly harvested cells, or cells that have been incubated in tissue culture, in the presence of a detectably labeled antibody capable of identifying fingerprint gene peptides, and detecting the bound antibody by any of a number of techniques well known in the art.

The biological sample may be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled gene-specific antibody. The solid phase support may then be washed with the buffer a second time to remove unbound antibody. The amount of bound label on solid support may then be detected by conventional means.

The terms “solid phase support or carrier” are intended to encompass any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present disclosure. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-wild-type or -mutant fingerprint gene peptide antibody may be determined according to well known methods. Those skilled in the art will be able to determine operative and optimal assay conditions for each determination by employing routine experimentation.

One of the ways in which the gene peptide-specific antibody can be detectably labeled is by linking the same to an enzyme and using it in an enzyme immunoassay (EIA) (Voller, Ric Clin Lab, 8:289-98 (1978) [“The Enzyme Linked Immunosorbent Assay (ELISA)”, Diagnostic Horizons 2: 1-7, 1978, Microbiological Associates Quarterly Publication, Walkersville, Md.]; Voller, et al., J. Clin. Pathol., 31:507-20 (1978); Butler, Meth. Enzymol., 73:482-523 (1981); Maggio (ed.), Enzyme Immunoassay, CRC Press, Boca Raton, Fla. (1980); Ishikawa, et al., (eds.) Enzyme Immunoassay, Igaku-Shoin, Tokyo (1981)). The enzyme that is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild-type, mutant, or expanded peptides through the use of a radioimmunoassay (RIA) (see, e.g. Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediamine-tetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present disclosure. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Throughout this application, various publications, patents and published patent applications are referred to by an identifying citation. The disclosures of these publications, patents and published patent specifications referenced in this application are hereby incorporated by reference into the present disclosure to more fully describe the state of the art to which this disclosure pertains.

The following examples are intended only to illustrate the present disclosure and should in no way be construed as limiting the subject disclosure.

### EXAMPLES

### Example 1

### Generation of Mice Comprising CTSG Disruptions

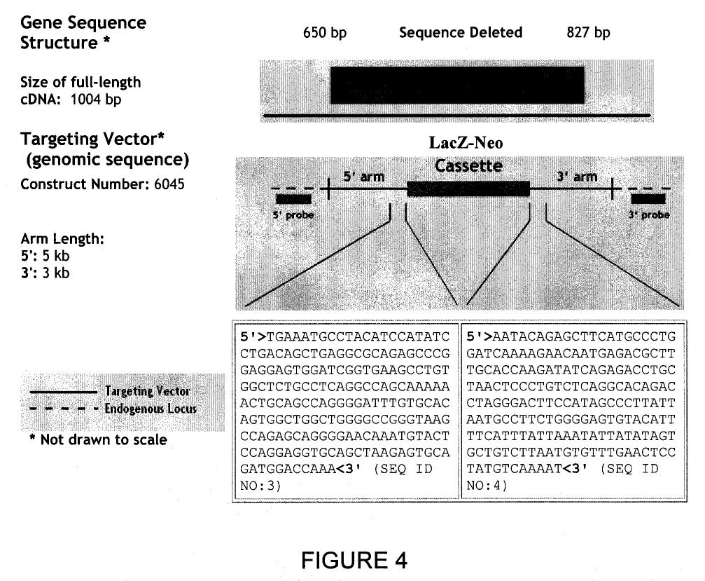

To investigate the role of CTSG, disruptions in CTSG genes were produced by homologous recombination. Specifically, transgenic mice comprising disruptions in CTSG genes were created. More particularly, as shown in FIG. 4, a CTSG-specific targeting construct based upon SEQ ID NO:1 or the sequence identified in Genebank as Accession No. NM_{—}007800; GI:6681082, was created using as the targeting arms (homologous sequences) in the construct the oligonucleotide sequences identified herein as SEQ ID NO:3 or SEQ ID NO:4.

The targeting construct was introduced into ES cells derived from the 129/OlaHsd mouse substrain to generate chimeric mice. The F1 mice were generated by breeding with C57BL/6 females, and the resultant F1N0 heterozygotes were backcrossed to C57BL/6 mice to generate F1N1 heterozygotes. The F2N1 homozygous mutant mice were produced by intercrossing F1N1 heterozygous males and females.

Genomic DNA from the recombinant ES line was assayed for homologous recombination using polymerase chain reactions (PCRs). Both 5′ PCR reconfirmation and 3′ PCR reconfirmation was performed. The method employed a gene-specific (GS) primer, which was outside of and adjacent to the targeting vector arm, paired in succession with one of three primers in the insertion fragment. The “DNA sample control” employed a primer pair intended to amplify a fragment from a non-targeted genomic locus. The “positive control” employed the GS primer paired with a primer at the other end of the arm. Amplified DNA fragments were visualized by ethidium bromide staining following agarose gel electrophoresis and matched the expected product sizes, in base pairs (bp).

In addition, genomic DNA isolated from both the parent ES line and the recombinant ES line was digested with restriction enzymes (determined to cut outside of the construct arms). The DNA was analyzed by Southern hybridization, and probed with a radiolabeled DNA fragment that hybridized outside of and adjacent to the construct arm. The parent ES line (negative control) showed bands representing the endogenous (wild-type) allele. In contrast, the recombinant ES line showed an additional band representing the targeted allele from the expected homologous recombination event.

The initial germ line F1 (129×C57BL/6) mice were genotyped by either PCR or Southern blot analysis. For both PCR and Southern analysis, oligonucleotides or probes were selected outside the targeting vector to avoid detecting vector alone and to confirm the homologous recombination event. F2 generation mice [F1(129×C57BL/6)×F1 (129×C57BL/6)] were subsequently genotyped by PCR analysis.

Gene expression analysis was performed using the knocked-in lacZ as a reporter gene and RT-PCR. In the case of lacZ expression assays some signals may not have been detected due to insertional silencing or insertional mutations.

### Example 2

### Expression Analysis

RT-PCR Expression. Total RNA was isolated from the organs or tissues from adult C57BL/6 wild-type mice. RNA was DNaseI treated, and reverse transcribed using random primers. The resulting cDNA was checked for the absence of genomic contamination using primers specific to non-transcribed genomic mouse DNA. cDNAs were balanced for concentration using HPRT primers.

High levels of RNA transcripts were detectable only in bone marrow. Very low levels of RNA transcripts were detectable in spleen and thymus. Whereas, no RNA transcripts were detectable in brain, cortex, subcortical region, cerebellum, brainstem, olfactory bulb, spinal cord, eye, Harderian glands, heart, lung, liver, pancreas, kidney, lymph nodes, skin, gallbladder, urinary bladder, pituitary gland, adrenal gland, salivary gland, skeletal muscle, tongue, stomach, small intestine, large intestine, cecum, testis, epididymis, seminal vesicle, coagulating gland, prostate gland, ovaries, uterus and white fat.

LacZ Reporter Gene Expression. In general, tissues from 7-12 week old heterozygous mutant mice were analyzed for lacZ expression. Organs from heterozygous mutant mice were frozen, sectioned (10 μm), stained and analyzed for lacZ expression using X-Gal as a substrate for beta-galactosidase, followed by a Nuclear Fast Red counterstaining.

In addition, for brain, wholemount staining was performed. The dissected brain was cut longitudinally, fixed and stained using X-Gal as the substrate for beta-galactosidase. The reaction was stopped by washing the brain in PBS and then fixed in PBS-buffered formaldehyde.

Wild-type control tissues were also stained for lacZ expression to reveal any background or signals due to endogenous beta-galactosidase activity. The following tissues can show staining in the wild-type control sections and are therefore not suitable for X-gal staining: small and large intestines, stomach, vas deferens and epididymis. It has been previously reported that these organs contain high levels of endogenous beta-galactosidase activity.

LacZ (beta-galactosidase) expression was not detectable in any of the tissues examined: brain, spinal cord, sciatic nerve, eyes, Harderian glands, thymus, spleen, lymph nodes, bone marrow, aorta, heart, lung, liver, gallbladder, pancreas, kidney, urinary bladder, trachea, larynx, pharynx, esophagus, thyroid gland, parathyroid gland, pituitary gland, adrenal glands, salivary glands, tongue, skeletal muscle, skin, male and female reproductive systems.

### Example 3

### Physical Examination

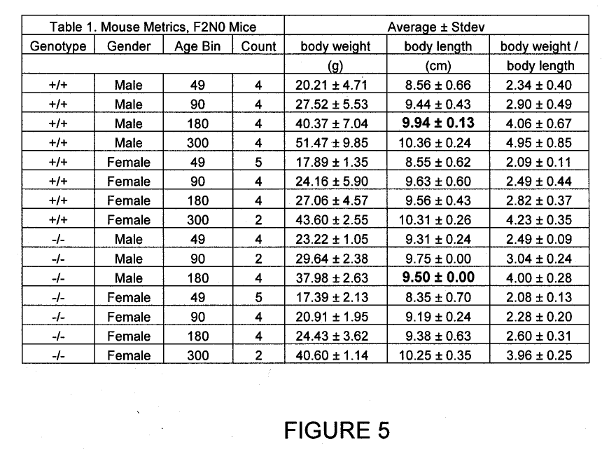

A complete physical examination was performed on each mouse. Mice were first observed in their home cages for a number of general characteristics including activity level, behavior toward siblings, posture, grooming, breathing pattern and sounds, and movement. General body condition and size were noted as well identifying characteristics including coat color, belly color, and eye color. Following a visual inspection of the mouse in the cage, the mouse was handled for a detailed, stepwise examination. The head was examined first, including eyes, ears, and nose, noting any discharge, malformations, or other abnormalities. Lymph nodes and glands of the head and neck were palpated. Skin, hair coat, axial and appendicular skeleton, and abdomen were also examined. The limbs and torso were examined visually and palpated for masses, malformations or other abnormalities. The anogenital region was examined for discharges, staining of hair, or other changes. If the mouse defecates during the examination, the feces were assessed for color and consistency. Abnormal behavior, movement, or physical changes may indicate abnormalities in general health, growth, metabolism, motor reflexes, sensory systems, or development of the central nervous system. Mouse body weights and body lengths were measured at 49, 90, 180 and 300 days of age. Mouse metrics data is shown in FIG. 5 (Table 1). When compared to wild-type control mice, certain homozygous mice exhibited decreased body length.

### Example 4

### Necropsy

Necropsy was performed on mice following deep general anesthesia, cardiac puncture for terminal blood collection, and euthanasia. Body lengths and body weights were recorded for each mouse. The necropsy included detailed examination of the whole mouse, the skinned carcass, skeleton, and all major organ systems. Lesions in organs and tissues were noted during the examination. Designated organs, from which extraneous fat and connective tissue have been removed, were weighed on a balance, and the weights were recorded. Weights were obtained for the following organs: heart, liver, spleen, thymus, kidneys, and testes/epididymides.

### Example 5

### Histopathological Analysis

Harvested organs were fixed in about 10% neutral buffered formalin for a minimum of about 48 hours at room temperature. Tissues were trimmed and samples taken to include the major features of each organ. If any abnormalities were noted at necropsy or at the time of tissue trimming, additional sample(s), if necessary, were taken to include the abnormalities so that it is available for microscopic analysis. Tissues were placed together, according to predetermined groupings, in tissue processing cassettes. All bones (and any calcified tissues) were decalcified with a formic acid or EDTA-based solution prior to trimming.

The infiltration of the tissues by paraffin was performed using an automated tissue processor. Steps in the cycle included dehydration through a graded series of ethanols, clearing using xylene or xylene substitute and infiltration with paraffin. Tissues were embedded in paraffin blocks with a standard orientation of specified tissues within each block. Sections were cut from each block at a thickness of about 3-5 μm and mounted onto glass slides. After drying, the slides were stained with hematoxylin and eosin (H&E) and a glass coverslip was mounted over the sections for examination. Histopathology examination was performed by board certified Veterinary Pathologists. One male and one female F2N1 300 day old homozygous mice were examined. The female 300 day old homozygous mouse exhibited lymphocytic infiltrate of the connective tissue of the salivary gland, degeneration of the thalamus in the cerebrum of the brain, lymphocytic infiltrate of the connective tissue of the pancreas, hyperplasia of the capsule of the adrenal gland, accessory cortical nodule of the pericapsular fat of the adrenal gland, dilation of the duct of the clitoral gland, degeneration of the brainstem, degeneration of the spinal cord, epidermal inclusion cyst of the spinal cord, predominantly lymphocytic infiltrate of the peribronchial of the lung, and lymphocytic infiltrate of the lamina propia of the urinary bladder. The male 300 day old homozygous mouse exhibited lymphocytic infiltrate of brown adipose tissue, acute inflammation of the aortic root of the heart, arteritis of the left ventricle of the heart, predominantly lymphocytic infiltrate of connective tissue of the pancreas, predominantly lymphocytic infiltrate of the periductal of the pancreas, acute inflammation of the glandular stomach, dilation of the glands of the mucosa of the glandular stomach, hyperplasia of the glands of the mucosa of the glandular stomach, increased extramedullary hematopoiesis of the red pulp of the spleen, hyperplasia of the lymph node, aspermia of the epididymis, chronic inflammation of the epididymis, lymphocytic infiltrate of the periprostatic fat of the prostate gland, degeneration of the seminiferous tubules of the testis, lymphocytic infiltrate of the bone of the mandible, chonic inflammation of transverse skeletal muscle, fresh hemorrhage of the brainstem, degeneration of the neuropil of the brainstem, mixed inflammation of the perineurium of the sciatic nerve, degeneration of the white matter of the spinal cord, mixed inflammation of the alveoli of the lung, predominantly lymphocytic infiltrate of the vasculature of the lung, hyalin droplet in the nasal epithelium, acute inflammation of the extraocular muscles of the eye, nephropathy of the cortex of the kidney, chronic inflammation of the arcuate artery vasculature of the kidney.

### Example 6

### Behavioral Analysis—Rotarod Test

The Accelerating Rotarod was used to screen for motor coordination, balance and ataxia phenotypes. Mice were allowed to move about on their wire-cage top for 30 seconds prior to testing to ensure awareness. Mice were placed on the stationary rod, facing away from the experimenter. The “speed profile” programs the rotarod to reach 60 rpm after six minutes. A photobeam was broken when the animal fell, which stopped the test clock for that chamber. The animals were tested over three trials with a 20-minute rest period between trials, after which the mice were returned to fresh cages. The data was analyzed to determine the average speed of the rotating rod at the fall time over the three trials. A decrease in the speed of the rotating rod at the time of fall compared to wild-types indicated decreased motor coordination possibly due to a motor neuron or inner ear disorder.

### Example 7

### Behavioral Analysis—Startle Test

The startle test screens for changes in the basic fundamental nervous system or muscle-related functions. The startle reflex is a short-latency response of the skeletal musculature elicited by a sudden auditory stimulus. This includes changes in 1) hearing—auditory processing; 2) sensory and motor processing—related to the auditory circuit and culminating in a motor related output; 3) global sensory changes; and motor abnormalities, including skeletal muscle or motor neuron related changes.

The startle test also screens for higher level cognitive functions. The startle reflex can be modulated by negative affective states like fear or stress. The cognitive changes include: 1) sensorimotor processing such as sensorimotor gating changes related to schizophrenia; 2) attention disorders; 3) anxiety disorders; and 4) thought disturbance disorders.

The mice were tested in a San Diego Instruments SR-LAB sound response chamber. Each mouse was exposed to 9 stimulus types that were repeated in pseudo-random order ten times during the course of the entire 25-minute test. The stimulus types in decibels were: p80, p90, p100, p110, p120, pp80, p120, pp90, p120, pp100, and p120; where p=40 msec pulse, pp=20 msec prepulse. The length of time between a prepulse and a pulse was 100 msec (onset to onset). The mean Vmax of the ten repetitions for each trial type was computed for each mouse.

### Example 8

### Behavioral Analysis—Hot Plate Test

The hot plate analgesia test was designed to indicate an animal's sensitivity to a painful stimulus. The mice were placed on a hot plate of about 55.5° C., one at a time, and latency of the mice to pick up and lick or fan a hindpaw was recorded. A built-in timer was started as soon as the subjects were placed on the hot plate surface. The timer was stopped the instant the animal lifted its paw from the plate, reacting to the discomfort. Animal reaction time was a measurement of the animal's resistance to pain. The time points to hindpaw licking or fanning, up to a maximum of about 60-seconds, was recorded. Once the behavior was observed, the animal was immediately removed from the hot plate to prevent discomfort or injury.

### Example 9

### Behavioral Analysis—Tail Flick Test

The tail-flick test is a test of acute nociception in which a high-intensity thermal stimulus is directed to the tail of the mouse. The time from onset of stimulation to a rapid flick/withdrawal from the heat source is recorded. This test produces a simple nociceptive reflex response that is an involuntary spinally mediated flexion reflex.

### Example 10

### Behavioral Analysis—Open Field Test

The Open Field Test was used to examine overall locomotion and anxiety levels in mice. Increases or decreases in total distance traveled over the test time are an indication of hyperactivity or hypoactivity, respectively.

The open field provides a novel environment that creates an approach-avoidance conflict situation in which the animal desires to explore, yet instinctively seeks to protect itself. The chamber is lighted in the center and has no places to hide other than the corners. A normal mouse typically spends more time in the corners and around the periphery than it does in the center. Normal mice however, will venture into the central regions as they explore the chamber. Anxious mice spend most of their time in the corners, with almost no exploration of the center, whereas bold mice travel more, and show less preference for the periphery versus the central regions of the chamber.

Each mouse was placed gently in the center of its assigned chamber. Tests were conducted for 10 minutes, with the experimenter out of the animals' sight. Immediately following the test session, the fecal boli were counted for each subject: increased boli are also an indication of anxiety. Activity of individual mice was recorded for the 10-minute test session and monitored by photobeam breaks in the x-, y- and z-axes. Measurements taken included total distance traveled, percent of session time spent in the central region of the test apparatus, and average velocity during the ambulatory episodes. Increases or decreases in total distance traveled over the test time indicate hyperactivity or hypoactivity, respectively. Alterations in the regional distribution of movement indicates anxiety phenotypes, i.e., increased anxiety if there is a decrease in the time spent in the central region.

### Example 11

### Behavioral Analysis—Metrazol Test

To screen for phenotypes involving changes in seizure susceptibility, the Metrazol Test was be used. About 5 mg/ml of Metrazol was infused through the tail vein of the mouse at a constant rate of about 0.375 ml/min. The infusion caused all mice to experience seizures. Those mice who entered the seizure stage the quickest were thought to be more prone to seizures in general.

The Metrazol test can also be used to screen for phenotypes related to epilepsy. Seven to ten adult wild-type and homozygote males were used. A fresh solution of about 5 mg/ml pentylenetetrazole in approximately 0.9% NaCl was prepared prior to testing. Mice were weighed and loosely held in a restrainer. After exposure to a heat lamp to dilate the tail vein, mice were continuously infused with the pentylenetetrazole solution using a syringe pump set at a constant flow rate. The following stages were recorded: first twitch (sometimes accompanied by a squeak), beginning of the tonic/clonic seizure, tonic extension and survival time. The dose required for each phase was determined and the latency to each phase was determined between genotypes. Alterations in any stage may indicate an overall imbalance in excitatory or inhibitory neurotransmitter levels.

### Example 12

### Behavioral Analysis—Tail Suspension Test

The tail suspension test is a single-trial test that measures a mouse's propensity towards depression. This method for testing antidepressants in mice was reported by Steru et al., (1985, Psychopharmacology 85(3):367-370) and is widely used as a test for a range of compounds including SSRI's, benzodiazepines, typical and atypical antipsychotics. It is believed that a depressive state can be elicited in laboratory animals by continuously subjecting them to aversive situations over which they have no control. It is reported that a condition of “learned helplessness” is eventually reached.

Mice were suspended on a metal hanger by the tail in an acoustically and visually isolated setting. Total immobility time during the six-minute test period was determined using a computer algorithm based upon measuring the force exerted by the mouse on the metal hanger. An increase in immobility time for mutant mice compared to wild-type mice may indicate increased “depression.” Animals that ceased struggling sooner may be more prone to depression. Studies have shown that the administration of antidepressants prior to testing increases the amount of time that animals struggle

### Example 13

### Hematological Analysis

Blood samples were collected via a terminal cardiac puncture in a syringe. About one hundred microliters of each whole blood sample were transferred into tubes pre-filled with EDTA. Approximately 25 microliters of the blood was placed onto a glass slide to prepare a peripheral blood smear. The blood smears were later stained with Wright's Stain that differentially stained white blood cell nuclei, granules and cytoplasm, and allowed the identification of different cell types. The slides were analyzed microscopically by counting and noting each cell type in a total of 100 white blood cells. The percentage of each of the cell types counted was then calculated. Red blood cell morphology was also evaluated.

Microscopic examinations of blood smears were performed to provide accurate differential blood leukocyte counts. The leukocyte differential counts were provided as the percentage composition of each cell type in the blood.

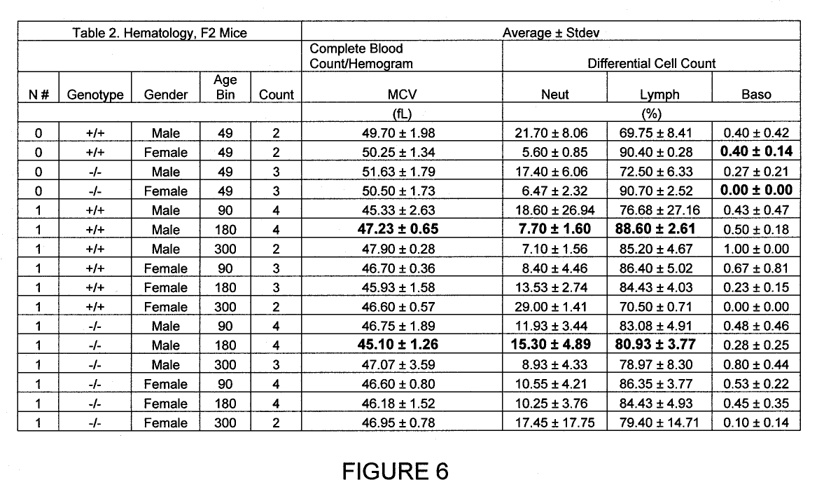

Interesting hematology data are shown in FIG. 6 (Table 2). When co pared to wild-type control mice, certain honozygous mice exhibited decreased mean corpuscular volume (MCV), increased neutrophils, decreased lymphocytes, and decreased basophils.

Mean corpuscular volume (MCV) reflects the size of red blood cells as the volume occupied by a single red blood cell. Increased MCV may indicate macrocytic anemia or B6 or folic acid deficiency. Decreased MCV may indicate microcytic anemia, and possible iron deficiency.

Neutrophils, also called granulocytes or segmented neutrophils, are the main defense against infection and antigens. High levels may indicate an active immune system, low levels may indicate a depressed immune system or low production by bone marrow.

Lymphocytes are involved in the protection of the body from viral infection. Elevated levels may indicate an active viral infection, and low levels may indicate an exhausted immune system.

Basophils are known to carry histamine, heparin and serotonin. High basophils are found in allergic reaction, low levels are normal.

### Example 14

### Serum Chemistry

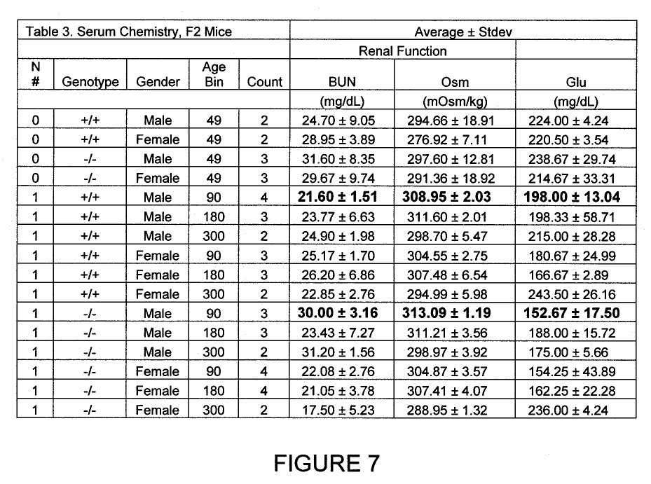

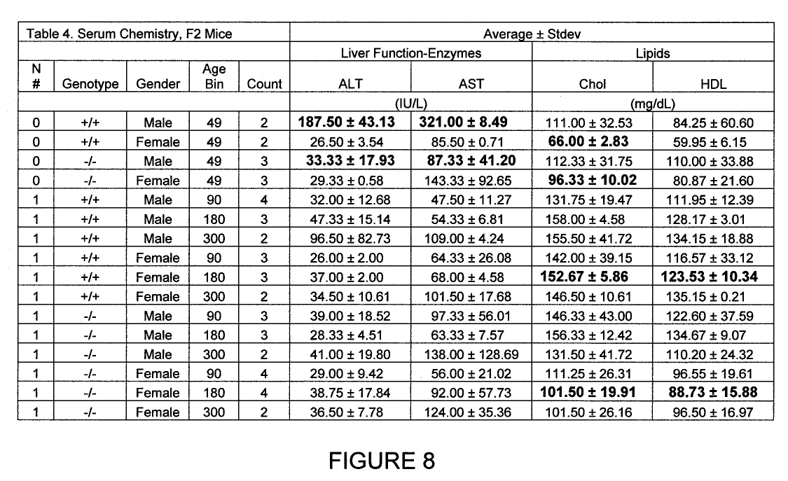

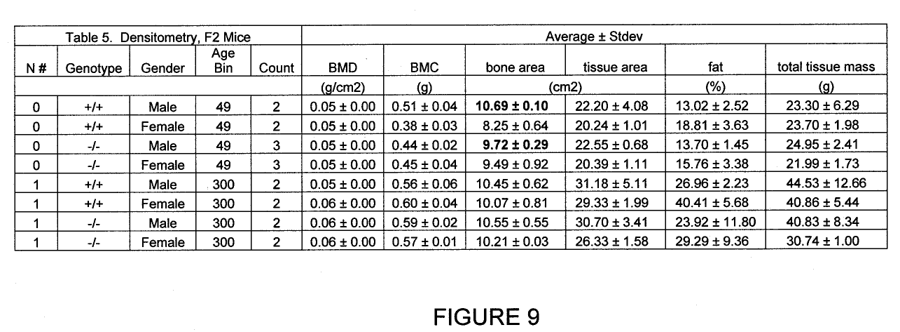

Homozygous mutant mice were compared with age- and gender-matched wild-type control mice. Non-terminal blood samples were collected via retro-orbital venous puncture in capillary tubes. This procedure supplied approximately 200 uL of whole blood that was transferred into a serum tube with a gel separator for serum chemistry analysis. The blood sample was converted to serum by centrifugation in a serum tube with a gel separator. Each serum sample was then analyzed as described below. Serum data were collected on a Roche/Hitachi 912 Automatic Analyzer using Boehringer Mannheim Corporation reagents. Serum samples were evaluated with a clinical chemistry panel and were evaluated for the following serum components: electrolytes (sodium (Na), potassium (K), chloride (Cl), bicarbonate (Bicarb)), liver function ((enzymes) alkaline phosphatase (ALP), alanine aminotransferase (ALT), aspartate aminotransferase (AST), lactate dehydrogenase (LD)), renal function tests (blood urea nitrogen (BUN), creatinine (Creat), osmolality (Osm), liver function ((other) protein, total (T Prot), albumin (Alb), globulin (Glob), bilirubin, total (Bil T)), inorganic ions (calcium (Ca), phosphorus (Phos)), lipid profile including cholesterol (Chol), high density lipoprotein (HDL), low density lipoprotein (LDL), triglycerides (TG), glucose (Glu) and creatine kinase (CK). Results for homozygous mice were compared to wild-type control mice with same ES parent, gender, F, N, and age. For all data collected, two-tailed pair-wise statistical significance was established using a Student t-test. Statistical significance was defined as P<0.05. Data were considered statistically significant if 1-p vs. wild-type control value was >0.95. Statistically significant serum chemistry phenotypes are displayed in bold in FIGS. 7 and 8 (Tables 3 and 4); average values, plus or minus the standard deviation, are shown for F2 mice.

When compared to wild-type control mice, certain homozygous mice exhibited increased blood urea nitrogen (BUN), increased osmolality (Osm), decreased glucose, decreased alanine aminotransferase (ALT), decreased aspartate aminotransferase (AST), abnormal cholesterol, and decreased high density lipoprotein (HDL).

BUN is the end product of protein metabolism and the BUN concentration is influence by the rate of excretion. Increases may be caused by excessive protein intake, kidney damage, low fluid intake, intestinal bleeding, exercise or heart failure. decreases may be seen in high carbohydrate/low protein diets, increased anabolic demand (such as pregnancy, infancy, acromegaly), malabsorption states and severe liver damage.

Serum glucose results from the digestion of carbohydrates and the conversion of glycogen by the liver. Glucose is the primary energy source for most cells. It is regulated by insulin, glucagon, thyroid hormone, liver enzymes and adrenal hormones. Increased fasting serum glucose may be indicative of diabetes mellitis.

Alanine aminotransferase (ALT) is a liver enzyme which also occurs in the kidneys, heart, and skeletal muscles. ALT is one of two main liver function blood serum tests. ALT is a marker of acute liver damage and is slightly to moderately elevated in any condition that produces acute liver cell injury, e.g. active cirrhosis and hepatitis.

Aspartate aminotransferase (AST) is one of two main liver function blood serum tests. AST levels fluctuate with the extent of cellular necrosis (cell death). Increased AST levels may be seen in any condition involving necrosis of hepatocytes, myocardial cells, or skeletal muscle cells.

AST level may be used to help detect a recent myocardial infarction and in differential diagnosis of acute hepatic disease.

Cholesterol is a structural component of cell membrane and plasma lipoproteins and is essential in the synthesis of steroid hormones, glucocorticoids, and bile acids. Low levels of cholesterol are seen in immune compromised patients, poor dietary habits, malabsorption, and liver or kidney disease.

HDL is the cholesterol carried by the alpha lipoproteins. HDL inhibits cellular uptake of LDL and is a carrier that removes cholesterol from the peripheral tissues and transports it back to the liver for catabolism and excretion.

Serum HDLs are a protective factor against cardiovascular diseases and disorders, such as, for example, coronary artery disease or atherosclerosis. Many epidemiological studies show a strong inverse relationship between serum HDL-cholesterol (HDL-C) and the likelihood of developing coronary artery disease. Frohlich J J., et. al., “The clinical significance of serum high density lipoproteins,” Clin. Biochem. 1989 December; 22(6):417-23. As such, the HDL levels of the mice of the present invention are analogous to a protective effect against human cardiovascular diseases or disorders, such as, for example coronary artery disease.

### Example 15

### Densitometric Analysis

Mice were euthanized and analyzed using a PIXImus™ densitometer. An x-ray source exposed the mice to a beam of both high and low energy x-rays. The ratio of attenuation of the high and low energies allowed the separation of bone from soft tissue, and, from within the tissue samples, lean and fat. Densitometric data including Bone Mineral Density (BMD presented as g/cm2), Bone Mineral Content (BMC in g), bone and tissue area, total tissue mass, and fat as a percent of body soft tissue (presented as fat %) were obtained and recorded.

Interesting densitometry data is shown in FIG. 8 (Table 5). When compared to wild-type control mice, certain homozygous mice exhibited decreased bone area.

### Example 16

### Development

Animals are genotyped using one of two methods. The first method uses the polymerase chain reaction (PCR) with target-specific and Neo primers to amplify DNA from the targeted gene. The second method uses PCR and Neo primers to “count” the number of Neo genes present per genome.

If homozygous mutant mice are not identified at weaning (3-4 weeks old), animals were assessed for lethality linked with the introduced mutation. This evaluation included embryonic, perinatal or juvenile death.

Newborn mice were genotyped 24-48 hours after birth and monitored closely for any signs of stress. Dead/dying pups were recorded and grossly inspected and if possible, genotyped. In the case of perinatal death, late gestation embryos (˜E19.5, i.e., 19.5 days post-coitum) or newborn pups were analyzed, genotyped and subject to further characterization.

If there was no evidence of perinatal or juvenile lethality, heterozygous mutant mice were set up for timed pregnancies. Routinely, E10.5 embryos are analyzed for gross abnormalities and genotyped. Depending on these findings, earlier (routinely >E8.5) or later embryonic stages are characterized to identify the approximate time of death. If no homozygous mutant progeny are detected, blastocysts (E3.5) are isolated, genotyped directly or grown for 6 days in culture and then genotyped. Any suspected genotype-related gross abnormalities are recorded.

### Example 17

### Fertility

The reproductive traits of male and female homozygous mutant mice are tested to identify potential defects in spermatogenesis, oogenesis, maternal ability to support pre- or post-embryonic development, or mammary gland defects and ability of the female knockout mice to nurse their pups.

Homozygous mutant (−/−) mice of each gender were set up in a fertility mating with either a wild-type (+/+) mate or a homozygous mutant mouse of the opposite gender at about seven to about ten weeks of age. The numbers of pups born from one to three litters were recorded at birth. Three weeks later, the live pups were counted and weaned.

Males and females were separated after they had produced two litters or at six months (26 weeks) of age, whichever comes first.

### Example 18

### Pain Analysis

Pain is one of the commonest symptoms of illness or tissue damage or a metabolic disturbance. The pain is noticeable when mechanical, thermal, chemical or electrical stimuli exceed a certain threshold value. More particularly, neuropathic pain, a sensory disorder that results from a variety of nerve injuries, infection, or cased by other diseases, occurs at a high prevalence and is a challenging medical condition. Thus, there is a need to identify new genes that are involved in the development of pain. To identify the role of CTSG in the development of pain, the following tests were conducted:

Formalin Test. The Formalin test for nociception involves injecting a noxious substance, about 3% Formalin solution, into the plantar surface of the mouse's hindpaw. The mouse reacts to the Formalin injection (by licking and flinching the injected hindpaw, for example). An automated system is used to detect the number of times the mouse flinches over a period of about one hour. The response to Formalin injection occurs as two distinct phases. Phase one occurs within about the first 10 minutes of the test and is thought to be the result of C-fiber activation due to the chemical stimulation of the nociceptors. Phase two occurs within about 11-60 minutes following the injection. Phase two appears to be due to a neurogenic inflammatory reaction within the injected paw and functional changes in the dorsal horn of the spinal cord.

Homozygous mutant (−/−) mice showing a difference in the response to Formalin, relative to wild-type control mice, may demonstrate a role of CTSG in nociception.

Paw Thermal Test. The nociception in the paw theremal test is the heat generated from a radiant bulb. About 12.5 μL of Complete Freund's Adjuvant (CFA) solution is injected into the plantar surface of a paw. After about 24 hours, mice are placed into test chambers and allowed to acclimate to the chamber for a minimum of about 30 minutes, or until exploratory and grooming behavior cease. A radiant bulb is positioned under a hind paw of the mouse, such that a focused light beam contacts the hind paw and delivers a heat stimulus. The mouse is observed for a response of either a stomp action or a sharp withdrawal of the paw. An automatic motion sensor stops the heat stimulus when the mouse responds. The response latency is recorded.

Homozygous mutant mice (−/−) that respond in less time (i.e., shorter latency to remove the paw) indicate an increased sensitivity to pain, or decreased pain threshold.

Mechanical Sensitivity Test. The nociception stimulus in the mechanical sensitivity test is the force of a filament applied to the plantar surface of both hind paws. About 12.5 μL of Complete Freund's Adjuvant (CFA) solution is injected into the plantar surface of a paw. After approximately 28 hours, mice are placed into test chambers and allowed to acclimate to the chamber for a minimum of about 30 minutes, or until exploratory and grooming behavior cease. A filament is then brought into contact with the paw. The filament touches the plantar surface of the hind paws and begins to exert an upward force below the threshold of feeling. The force increased at a rate of about 0.25 grams per second until the mouse removes his hindpaw or until the maximum force of about 5.0 grams is reached in approximately 20 seconds. The latency for the mouse to remove the hindpaw is recorded.

Homozygous mutant mice (−/−) that respond in less time (i.e., shorter latency to remove the paw) may have an increased sensitivity to pain, or decreased pain threshold, whereas homozygous mice exhibiting an increased latency to withdraw the paw may have a decreased sensitivity to pain, or an increased pain threshold.

Mice having a disruption in the CTSG gene, according to the present disclosure, may be used to screen for nociceptive agents and to evaluate known compounds useful for treating pain.

### Example 19

### CTSG In Inflammatory Bowel Disease

Inflammatory bowel diseases (IBD) refer to diseases involved in the inflammation of the intestines. Chronic IBD involves aspects of both the innate and adaptive immune response, in that initial destruction of intestinal tissue during acute inflammation leads to a chronic T-cell mediated autoimmune disorder.

Crohn's disease is one major type of IBD. Crohn's disease typically occurs in the lowest portion of the small intestine (ileum), and the large intestine (colon or bowel), but it can occur in other parts of the digestive tract. Crohn's usually involves all layers of the intestinal wall. The disease can be difficult to diagnose because its symptoms, which include chronic diarrhea, crampy abdominal pain, loss of appetite, and weight loss, often mimic those of the other IBD type—ulcerative colitis—which affects only the colon.

According to the Crohn's and Colitis Foundation of America (CCFA), it is estimated that the incidence of Crohn's disease is from about 1.2 cases to about 15 cases per 100,000 people in the United States. While it can affect any age group, the onset of the disease most commonly occurs between the ages of 15 and 30, and between the ages of 60 and 80. Current treatment regimens, which include steroid treatment and immunosuppressives can ameliorate symptoms in patients; however, a high incidence of relapse and detrimental side effects suggest these treatments are less than ideal. Thus, there is a need in the art to identify therapeutically relevant targets involved in IBD etiology and progression.

To examine the role and function of CTSG in IBD, the following procedure was performed:

Procedure: Female wild-type and homozygous mutant mice about 10-12 weeks of age were fed dextran sulfate sodium (DSS) orally in drinking water for 7 days (days 1-7) followed by 7 days of water alone (days 8-14). A second 7-day course of DSS was then given (days 15-21). The dose of DSS used was either no DSS (control) or about 2%-3% (weight/volume) DSS. Weight was monitored daily during the course of the study. Stool samples were analyzed for consistency (normal, loose or diarrhea) and were tested for hemoccult positivity or gross bleeding. The mice were analyzed daily for the presence of rectal bleeding. On day 22, following the second course of DSS, mice were sacrificed for necropsy and histological analysis of the small and large intestines. Results of the observations, necropsy and histological analysis were used to determine if a disruption in the target gene results in changes in sensitivity or resistance to disease progression in response to DSS.

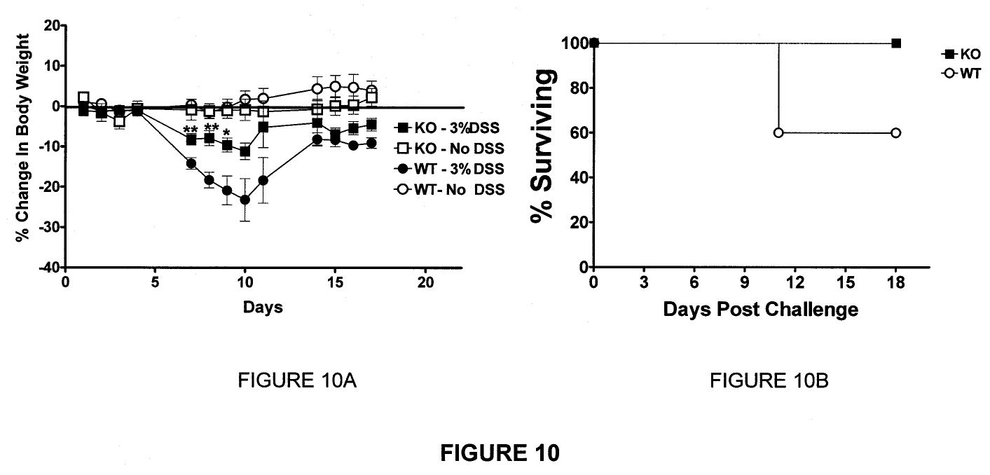

Results: Homozygous mice were evaluated for body weight and survival rate during the course of treatment with 3% DSS as describe above. Homozygous mice (−/−, n=6) and wild-type mice (+/+, n=5) were treated with 3% DSS, and control groups (−/−, n=9; +/+, n=6) were left untreated. Mice were analyzed as described. As shown in FIG. 10, homozygous mice (labeled KO in FIGS. 10A-10B) exhibited a decreased change in body weight in response to DSS treatment, relative to DSS treated controls (WT). In addition, as shown in FIG. 10B homozygous mice (KO) also exhibited increased survival rate, as indicated in the figure (p<0.05).

### Example 20

### Antigen Induced Airway Hyperactivity Model for Asthma

More than 17 million Americans are estimated to have asthma and the prevalence of the disease has increased 75% in the last 15 years. Acute asthmatic attacks are generally treated by bronchodilators, while preventative treatments included anti-inflammatory agents such as corticosteroids and, more recently, leukotriene modifiers. Like other allergies, asthma is the result of a hyperactive response by the immune system against an otherwise harmless protein (allergen; e.g. ragweed pollen) that includes infiltration of lung tissue by mast cells and eosinophils. Upon subsequent encounters with the allergen, an immediate hypersensitivity response occurs which results in impaired breathing and mucus formation.

Immunization: Female wild-type and homozygous mutant mice about 8-12 weeks of age were used. For each mouse type (wild-type or homozygous mutant), about 8 mice will be immunized with ovalbumin and about 8 will be sham immunized (non-sensitized) as controls for immunizations and aerosol challenge. To induce antigen specific airway hypersensitivity, mice are immunized by intraperitoneal (i.p.) injection of 20 micrograms of purified ovalbumin emulsified with 2 milligrams of the adjuvant aluminum hydroxide in a solution of phosphate buffered saline (PBS) on days 0 and 14. Total volume of the immunogen/adjuvant is 0.2 milliliters. Non-sensitized mice receive injections of aluminum hydroxide without ovalbumin.

Aerosol Challenge: On days 28, 29, and 30, mice are given an aerosol challenge of ovalbumin by placing mice in a plastic chamber and administering an aerosol of 1% ovalbumin (weight/volume) in PBS created by ultrasonic nebulization. The aerosol is administered for 20 minutes each day. Non-sensitized mice receive aerosolized PBS without ovalbumin.

Blood Collection: Blood is collected at day 0 prior to immunization, day 15 and day 31 following plethysmography (see below). Blood (150 microliters) is collected by a tail vein nick. Blood is analyzed for the presence of IgE, and the amount of IgE present that is specific for ovalbumin.

Measurement of Airway Hyperactivity: On day 31, airway activity is assessed by a non-invasive method, which measures breathing patterns in unrestrained animals. Mice are placed into a whole body plethysmograph and baseline readings of breathing patterns are taken for three minutes. Mice are exposed to nebulized PBS or methacholine (in a PBS solution) at doses ranging from three to 50 milligrams per milliliter. Methacholine acts to induce bronchoconstriction in a dose dependent manner by activation of effector cells (predominantly eosinophils) that migrate to lung tissue following aerosol challenge with ovalbumin or that may be constituitively present as a result of a disruption in the target gene. Doses (4-5 total) are administered for three minutes each and breathing patterns are recorded for five minutes following each dose of inhaled methacholine or vehicle. Following the final recording of breathing patterns, mice are euthanized by CO₂ prior to necropsy. Changes in the response to methacholine challenge in non-sensitized homozygous mutant mice relative to wild-type mice may be indicative of chronic obstructive pulmonary disease (COPD). Changes in the response to methacholine challenge in sensitized homozygous mutant mice relative to wild-type mice may be indicative of asthma.

### Example 21

### Cutaneous Allergy Model (aka Contact Dermatitis)

Cutaneous allergy is common in developed countries. Ten percent of U.S. children have allergic skin reactions such as dermatitis and cutaneous allergy represents the second most common occupational disease affecting about 75 in 100,000 workers. Contact allergy can be broadly categorized as allergic contact dermatitis (ACD), requiring multiple exposures to the allergen, and irritant contact dermatitis (ICD), which occurs following an initial exposure. Both ACD and ICD involve a local inflammatory response mediated by leukocytes such as neutrophils, monocytes, macrophages and mast cells. Unlike ICD, ACD also involves a T-cell component since the affected individual must be exposed to the allergen more than one time. These inflammatory reactions involve a complex interplay between blood borne cells, blood vessels and the affected tissue itself.

Allergic Contact Dermatitis (ACD)—Procedure: On day 0, wild-type and homozygous mutant mice have their abdomen shaved and are primed with the allergen, a 1% (weight/volume) solution of fluorescein isolthiocyanate (FITC; in acetone). Priming is performed by placing 100 microliters of the FITC solution on the shaved abdomen and 5 microliters of the solution on each footpad. On day 6, the ear thickness of each mouse is measured with vernier calipers or an engineer's micrometer to establish a baseline ear thickness. To each mouse 10 microliters of a 0.5% FITC (in acetone) solution is placed on both sides of the right ear. The acetone solution (without FITC) is added to the left ear. Ear thickness is measured again on day 7, and the mice are sacrificed for tissue harvest. Ear swelling is estimated by determining the change in ear thickness from baseline to 24 hours post-challenge. Ear swelling is indicative of an allergic response to the antigen. Histological changes, including changes in the cell types infiltrating the ear, may also be looked at to see changes in the type of inflammation. Other means of measuring inflammation, such as thermogenic imaging to gauge temperature changes, may also be used.

Irritant Contact Dermatitis (ICD): On day 0, the ear thickness of each mouse (wild-type and homozygous mutant) is measured. About ten microliters of an approximately 2% croton oil (volume/volume) solution (in a 4:1 acetone:olive oil base) is applied to about the top third of one ear (ispilateral ear), of restrained animals. About ten microliters of an approximately 4:1 acetone:olive oil solution is applied to about the top third of the other, control ear (contralateral ear). On day 7, the ear thickness of each mouse is measured, and the mice are sacrificed for tissue harvest. Ear swelling is estimated as above (see ACD procedure).

### Example 22

### Peritonitis Model

A major hallmark of inflammatory responses is the influx of neutrophils and monocytes to sites of tissue damage or infection. This process involves the interplay of cytokines and chemokines produced by non-hematopoietic cells and cognate receptors on leukocytes. Once attracted, these leukocytes become activated releasing still more cytokines and chemokines which induce most aspects of an inflammatory response. In order to more fully understand the pathways involved in the initial steps of inflammation, and to discover therapeutically relevant intervention points, animal models of inflammation are necessary. In a mouse model of peritonitis, mice are given an injection of thioglycollate broth (a yeast protein based media) to induce peritoneal inflammation. This site of inflammation allows easy isolation of infiltrating cells for in vitro study. In this study some aspects of the early inflammatory process, namely neutrophil recruitment and macrophage activation, in wildtype and gene deficient animals are compared. Procedure: Thioglycollate (Sigma) was reconstituted to 3% weight/volume in sterile water. Mice were administered 1 milliliter of the thioglycollate solution or an equal volume of sterile water (for control mice) i.p. At 1 or 3 days post thioglycollate challenge, mice are sacrificed. Some aspects of the early inflammatory process, namely neutrophil recruitment and macrophage activation were compared in homozygous mice relative to wild-type mice.

### Example 23

### Model for Metastatic Spread

Metastatic disease is the major cause of death for most cancer types. Metastasis is a complex multi-step process involving tumor homing to target organs, extravasation across endothelial barriers, invasion of organ tissue and tissue remodeling. The molecular mechanisms governing metastatic spread are poorly understood but certainly involve gene expression by target organ tissue and endothelium as well as the tumor itself.

A disease state is induced in mice of about 8-12 weeks of age, by intravenous administration of about 1×10⁶ viable tumor cells in the lateral tail vein. Tumor cells are grown in medium consisting Roswell Park Medical Institute (RPMI) 1640 media supplemented with about 10% fetal bovine serum (FBS) and antibiotics, are harvested under sterile conditions by trypsinazation and washed 3 times in Hank's balanced salt solution (HBSS) no more than about 60 minutes prior to injection. In order to dilate the lateral tail vein, mice are gently warmed by exposing a low wattage lamp to the cage just prior to injection. Mice are then placed in a restrainer and tumor cells injected (V_{T}=0.2 ml) into the lateral tail vein using a 1 cc syringe and 27 gauge needle. Immediately following injection, mice are removed from the restrainer and returned to their cage.

Mice are monitored daily for tumor progression on the skin and checked for general appearance, mobility, weight and ability to access food and water. Mice are sacrificed about 21 days after tumor challenge unless they appear moribund or display cachexia prior to this time. All mice are placed into necropsy following euthanasia by CO₂.

Homozygous mutant (−/−) mice of the present disclosure exhibiting a decrease in the number and spread of tumors (relative to wild-type controls) may indicate a role of CTSG in metastasis. Transgenic mice exhibiting a disruption in CTSG may be used to screen for putative agents and other known compounds for their treatment of cancer.

### Example 24

### CTSG in Arthritis

Transgenic mice of the present disclosure were tested in a model of rheumatoid arthritis to investigate the role of CTSG in the disease. Arthritis was induced by the intravenous (i.v.) administration of a cocktail of monoclonal antibodies (mAbs) specific for arthrogenic epitopes of type CII collagen. These antibodies were purchased as a kit from Chemicon and are specific to peptide fragments derived from the CB11 region of type II collagen (Cat # ECM1400). Twenty-four hours after each injection of mAbs, mice received an intraperontoneal (i.p.) administration (SOP # 00023) of about 25 μg lipopolysaccharide (LPS, Sigma), which served to boost the arthrogenic response of the mAbs. In the model validation study, mice received about 8 mg of mAbs as either a single administration or as two administrations of about 4 mg each spaced about 48 hr apart.

Disease monitoring: Mice were monitored daily following the first injection of mAbs for signs of articular inflammation. Disease severity was scored based upon the number of joints involved and the severity of inflammation (i.e. presence of redness, joint swelling, # of digits involved). Mice were sacrificed 7 days after the first administration of mAbs. This time point was chosen based on mAb manufacturer's recommendation and personal communication with independent researchers (J. Mobley, Pfizer). At 7 days, incidence of inflammation was 100% and was prior to onset of disease resolution. Mice were sacrificed with CO₂.

Results: Homozygous mice exhibited resistance to joint inflammation induced by arthritogenic antibodies. Specifically homozygous mice (KO) were demonstrated decreased disease severity in this model of rheumatoid arthritis as to their wild-type (WT) counterparts.

These results indicate that CTSG may provide a valuable target for the identification of therapeutic agents for the treatment of rheumatoid arthritis. As shown by these results, antagonism of CTSG may provide resistance to or treatment of the joint inflammation characteristic of rheumatoid arthritis.

### Example 25

### Septic Shock

Septicemia is the 9^{th} leading cause of death in the U.S. and sepsis in newborns represents a significant mediator of infant mortality. Septicemia is mediated by the pleiotropic effects of several inflammatory cytokines including TNF-α, IL-1α and IL-6 on multiple organ systems throughout the body. Septicemia is induced by the activation of monocytes and macrophages by systemic exposure to bacteria an bacterial cell wall products. The release of inflammatory cytokines by macrophages resident in blood and the peritoneal cavity is a common feature of most inflammatory responses. Therefore, delineating pathways critical to the release of these cytokines and finding targets that inhibit this process may have therapeutic value to a multitude of other diseases besides sepsis. In this model, mice will receive a challenge of a bacterial cell wall component, lipopolysaccharide (LPS), and the expression of inflammatory cytokines in serum will be measured in order to determine the role specific targets play in the initial inflammatory cascades involved in septic shock.

LPS challenge. Lipopolysaccharide (LPS) (Sigma) will be reconstituted to 1 mg/ml in PBS prior to administration to mice. Mice will receive 100 ug of LPS (in 0.1 ml volume) or an equal volume of PBS (control groups) i.p. using a 27 gauge needle and hand held restraint. Following injection the mice will be placed back into their cage. Separate cohorts of mice will have cardiac punctures performed following lethally dosed, avatine anesthesia at 1.5 and 6 hr post challenge. These time points have been chosen in order to capture the peak serum levels of specific inflammatory cytokines. Cardiac punctures will be performed on the mice.

The dose of LPS is considered a lethal dose for mice of the C57B1/6 background, but death does not occur until 24 hr post challenge. Therefore, no mice are expected to succumb to the LPS during the time course of this experiment. However, LPS does induce fever in mice within 6 hours. However, in order to detect serum levels of inflammatory cytokines, including IL-1α, which induces the fever response, blood is collected 6 hr post challenge. Following cardiac puncture, the mice will be immediately sacrificed by cervical dislocation.

### Example 26

### Cytofluorometric Analyses

Cells from thymus, lymph nodes, and spleen tissues were isolated from wild type and mutant mice and dispersed into a single cell suspension. The red blood cells were removed by lysis with Tris/NH₄Cl solution for 5 minutes at room temperature. The cell suspension was filtered with a nylon mesh and washed twice with a staining medium, which was HBSS with reduced phenol red, sodium azide, BSA, and EDTA 0.5×10⁶ cells/25 μl/staining were incubated with 1 μg/10 μl/staining of PE- or FITC-labeled antibodies (PharMingen, San Diego, Calif.) for 15 minutes on ice, washed once and fixed with 0.5% formamide in staining medium. Cytometric analyses were performed using FACScan (Becton Dickinson) as described previously (Hanna Z et al., Mol. Cell. Biol., 1994, 14:1084-1094). About 20,000 cells were recorded in each staining.

Interesting FACS data is shown in Table 6. When compared to wild-type control mice, homozygous mice exhibited significantly decreased CD4+CD25+ positive spleen cells and decreased CD8+ positive spleen cells.

TABLE 6
FACS-Spleen, F2N0 Mice

| | |
|---|---|
| CD4 + CD25+ | CD8+ |

| | | |
|---|---|---|
| Genotype | Count | (%) |

| | | | |
|---|---|---|---|
| +/+ | 4 | 2.66 ± 0.61 | 13.52 ± 2.29 |
| −/− | 6 | 1.97 ± 0.30 | 9.64 ± 0.97 |

| | | |
|---|---|---|
| 1-p vs. WT Control | 0.96 | 0.99 |

Immune system responses depend on T cells (dependent on the thymus gland for function) and B cells (which ultimately differentiate into antibody secreting plasma cells). These cells are present in bone marrow, lymphoid organs (i.e. thymus, lymph nodes, tonsils, spleen, etc) and peripheral blood. These lymphoid cells can be categorized by the presence of specific cell surface markers called clusters of differentiation (CD). Each CD marker is given a specific number and is found on certain cell types. Thus the function of a cell can often be predicted by the specific CD marker present. For example, all mature human T cells have CD3 on their cell surface (thus they are CD3+). In contrast, human B cells are CD19+ but CD3−. CD3+ T cells can further be divided by function. T cells capable of killing target cells (called cytotoxic T cells—CTL) and/or downregulating immune responses (called suppressor T cells) are CD8+. T cells that help B cells make antibody or other T cells (such as CTL) become active are called helper T cells (TH) and are CD4+. Soluble peptides secreted by activated TH turn on selective portions of the immune response. These immune mediator glycoproteins are produced by many different cell types and are termed cytokines.

Liu et al., (J. Translational Medicine, 2005, 3:5 (2005)) suggest strategies of antitumor immunotherapy by manipulation of CD4+ CD25+ regulatory T lymphocytes. Ghiringhelli et al., (Eur. J. Immunol. 34(2):336-344 (2004)) demonstrated the role of CD4+ CD25+ regulatory T cells in tumor-induced immune tolerance and the interest of regulatory T cell depletion to sensitize established tumors to immunotherapy. CD4+CD25+ cell expression may be exploited to provide insight to disease mechanism, diagnosis and treatment of organ-specific autoimmune diseases such as gastritis, oophoritis, orchitis, thyroiditis, and pancreatitis; as well as suppression of tumor immunity.

CD8+ cells play an important role in controlling various infections in animals and humans including CMV retinitis, toxoplasmosis, hepatitis B and C, herpes virus and HIV.

As is apparent to one of skill in the art, various modifications of the above embodiments can be made without departing from the spirit and scope of this disclosure. These modifications and variations are within the scope of this disclosure.

We claim:

1. A transgenic mouse whose genome comprises a homozygous disruption of the CTSG gene, wherein said mouse exhibits a phenotypic abnormality relative to a wild-type control mouse.

2. The transgenic mouse of claim 1, wherein the transgenic mouse exhibits, relative to a wild-type control mouse, a physical phenotypic abnormality comprising decreased body length.

3. The transgenic mouse of claim 1, wherein the transgenic mouse exhibits, relative to a wild-type control mouse, at least one histopathology phenotypic abnormality selected from the group consisting of lymphocytic infiltrate of the connective tissue of the salivary gland, degeneration of the thalamus in the cerebrum of the brain, lymphocytic infiltrate of the connective tissue of the pancreas, hyperplasia of the capsule of the adrenal gland, accessory cortical nodule of the pericapsular fat of the adrenal gland, dilation of the duct of the clitoral gland, degeneration of the brainstem, degeneration of the spinal cord, epidermal inclusion cyst of the spinal cord, predominantly lymphocytic infiltrate of the peribronchial of the lung, lymphocytic infiltrate of the lamina propia of the urinary bladder, lymphocytic infiltrate of brown adipose tissue, acute inflammation of the aortic root of the heart, arteritis of the left ventricle of the heart, predominantly lymphocytic infiltrate of connective tissue of the pancreas, predominantly lymphocytic infiltrate of the periductal of the pancreas, acute inflammation of the glandular stomach, dilation of the glands of the mucosa of the glandular stomach, hyperplasia of the glands of the mucosa of the glandular stomach, increased extramedullary hematopoiesis of the red pulp of the spleen, hyperplasia of the lymph node, aspermia of the epididymis, chronic inflammation of the epididymis, lymphocytic infiltrate of the periprostatic fat of the prostate gland, degeneration of the seminiferous tubules of the testis, lymphocytic infiltrate of the bone of the mandible, chonic inflammation of transverse skeletal muscle, fresh hemorrhage of the brainstem, degeneration of the neuropil of the brainstem, mixed inflammation of the perineurium of the sciatic nerve, degeneration of the white matter of the spinal cord, mixed inflammation of the alveoli of the lung, predominantly lymphocytic infiltrate of the vasculature of the lung, hyalin droplet in the nasal epithelium, acute inflammation of the extraocular muscles of the eye, nephropathy of the cortex of the kidney, chronic inflammation of the arcuate artery vasculature of the kidney.

4. The transgenic mouse of claim 1, wherein the transgenic mouse exhibits, relative to a wild-type control mouse, at least one hematological phenotypic abnormality selected from the group consisting of exhibited decreased mean corpuscular volume (MCV), increased neutrophils, decreased lymphocytes, and decreased basophils.

5. The transgenic mouse of claim 1, wherein the transgenic mouse exhibits, relative to a wild-type control mouse, at least one serum chemistry phenotypic abnormality selected from the group consisting of increased blood urea nitrogen (BUN), increased osmolality (Osm), decreased glucose, decreased alanine aminotransferase (ALT), decreased aspartate aminotransferase (AST), abnormal cholesterol, and decreased high density lipoprotein (HDL).

6. The transgenic mouse of claim 1, wherein the transgenic mouse exhibits, relative to a wild-type control mouse, at least one densitometric phenotypic abnormality selected from the group consisting of decreased bone area.

7. The transgenic mouse of claim 1, wherein the transgenic mouse exhibits, relative to a wild-type control mouse, at least one immunologic phenotypic abnormality selected from the group consisting of decreased decreased change in body weight in response to DSS treatment in an irritable bowel syndrome assay, and increased survival in an irritable bowel syndrome assay.

8. The transgenic mouse of claim 1, wherein the transgenic mouse exhibits, relative to a wild-type control mouse, at least one cytoflurometric assay (FACS) phenotypic abnormality selected from the group consisting of decreased CD4+CD25+ positive spleen cells and decreased CD8+ positive spleen cells.

9. A method of producing the transgenic mouse of claim 1, the method comprising:
a. providing a mouse stem cell comprising a disruption in the endogenous CTSG gene;
b. introducing the mouse stem cell into a blastocyst;
c. introducing the blastocyst into a pseudopregnant mouse, wherein the pseudopregnant mouse generates chimeric mice; and
d. breeding said chimeric mice to produce the transgenic mouse.

10. A cell or tissue isolated from the transgenic mouse of claim 1.

11. A targeting construct comprising:
a. a first polynucleotide sequence homologous to at least a first portion of the endogenous CTSG gene;
b. a second polynucleotide sequence homologous to at least a second portion of the CTSG gene; and
c. a gene encoding a selectable marker located between the first and second polynucleotide sequences.

12. A method of identifying an agent capable of modulating activity of a CTSG gene or of a CTSG gene expression product, the method comprising:
a. administering a putative agent to the transgenic mouse of claim 1;
b. administering the agent to a wild-type control mouse; and
c. comparing a physiological response of the transgenic mouse with that of the control mouse;
wherein a difference in the physiological response between the transgenic mouse and the control mouse is an indication that the agent is capable of modulating activity of the gene or gene expression product.

13. A transgenic mouse whose genome comprises a disruption in the endogenous CTSG gene, wherein said gene encodes for mRNA corresponding to the cDNA sequence of SEQ ID NO: 1, and wherein said disruption comprises replacement of nucleotides 650 to 827 of SEQ ID NO: 1 with a LacZ-Neo cassette.

14. A transgenic mouse whose genome comprises a null allele of the endogenous CTSG gene.


SEQUENCE LISTING (4 sequences)

SEQ ID NO: 1 - DNA, 1003 bp, Mus musculus
atgaaacctttcacacgagcaggaaaatagatctgtgacctctcagaaccacccttccttcctctctctccaggggaggataaaagtctggcagagaaaagccccggcagcaactgactaagcaacggttctggaaagatgcagcctctcctgctcctgttgacctttattctactccaaggagatgaggcagggaagatcattggaggccgagaagccaggccccattcctacccctacatggcatttcttctgatccagagtccagaagggctgagtgcttgtggagggtttctggtgcgagaagacttcgtcctaacagcagctcattgcttgggaagctccataaatgttactctaggggcccacaacatccaaatgcgagaaaggacccagcaactcatcacagtgctcagagccatccgccatcctgattacaatccgcagaacatccggaatgacatcatgcttctgcagctgaggcgcagagcccggaggagtggatcggtgaagcctgtggctctgcctcaggccagcaaaaaactgcagccaggggatttgtgcacagtggctggctggggccgggtaagccagagcaggggaacaaatgtactccaggaggtgcagctaagagtgcagatggaccaaatgtgcgccaatcgcttccagttctacaacagccagactcagatctgtgtggaaacccgagagaaaggaagtctgccttcaggggtgattctggtggccccctggtatgtagcaatgtggcccagggcatcgtctcctatggaagcaacaatggtaaccctccagctgtattcaccaaaatccagagcttcatgccctggatcaaaagaacaatgagacgctttgcaccaagatatcagagacctgctaactccctgtctcaggcacagacctagggacttccatagcccttattaatgccttctggggagtgtacattttcatttattaaatattatatagtgctgtcttaaaa

SEQ ID NO: 2 - protein, 260 aa, Mus musculus
MQPLLLLLTFILLQGDEAGKIIGGREARPHSYPYMAFLLIQSPEGLSACGGFLVREDFVLTAAHCLGSSINVTLGAHNIQMRERTQQLITVLRAIRHPDYNPQNIRNDIMLLQLRRRARRSGSVKPVALPQASKKLQPGDLCTVAGWGRVSQSRGTNVLQEVQLRVQMDQMCANRFQFYNSQTQICVGNPRERKSAFRGDSGGPLVCSNVAQGIVSYGSNNGNPPAVFTKIQSFMPWIKRTMRRFAPRYQRPANSLQAQT

SEQ ID NO: 3 - DNA, 200 bp, Artificial Sequence
  Targeting Vector
tgaaatgcctacatccatatcctgacagctgaggcgcagagcccggaggagtggatcggtgaagcctgtggctctgcctcaggccagcaaaaaactgcagccaggggatttgtgcacagtggctggctggggccgggtaagccagagcaggggaacaaatgtactccaggaggtgcagctaagagtgcagatggaccaaa

SEQ ID NO: 4 - DNA, 200 bp, Artificial Sequence
  Targeting Vector
aatacagagcttcatgccctggatcaaaagaacaatgagacgctttgcaccaagatatcagagacctgctaactccctgtctcaggcacagacctagggacttccatagcccttattaatgccttctggggagtgtacattttcatttattaaatattatatagtgctgtcttaatgtgtttgaactcctatgtcaaaat